(19) United States
(12) Patent Application Publication
(10) Pub. No.: US 2026/0069319 A1
(43) Pub. Date: Mar. 12, 2026

(54) WIRELESSLY POWERED IMPLANTABLE MEDICAL DEVICE SYSTEM AND METHODS THEREOF

(71) Applicants: Victor A. Lavi, Boston, MA (US); Yves A. Moser, Le Landeron (CH); Florian D. Böckle, Péry (CH); Noah Hamlin, Harrison, TN (US); David Sonnenshein, Somerville, MA (US); Christian M. Albertelli, Pelham, NH (US); Sam B. Paniccia, Brooklyn, NY (US); James Coughlin, Ipswich, MA (US); Frank Zizzo, Woodcliff Lake, NJ (US); Matthew L. Rensberger, Syracuse, IN (US)

(72) Inventors: Victor A. Lavi, Boston, MA (US); Yves A. Moser, Le Landeron (CH); Florian D. Böckle, Péry (CH); Noah Hamlin, Harrison, TN (US); David Sonnenshein, Somerville, MA (US); Christian M. Albertelli, Pelham, NH (US); Sam B. Paniccia, Brooklyn, NY (US); James Coughlin, Ipswich, MA (US); Frank Zizzo, Woodcliff Lake, NJ (US); Matthew L. Rensberger, Syracuse, IN (US)

(21) Appl. No.: 19/322,083

(22) Filed: Sep. 8, 2025

Related U.S. Application Data
(60) Provisional application No. 63/691,828, filed on Sep. 6, 2024.

(51) Int. Cl. A61B 17/70; A61B 17/00; A61B 17/68; A61L 2/20

(52) U.S. Cl. CPC A61B 17/7002; A61L 2/20; A61B 2017/00221; A61B 2017/00398; A61B 2017/00411; A61B 2017/00477; A61B 2017/681; A61L 2103/15

(57) ABSTRACT

An implantable medical device system includes an implantable medical device and an external controller configured for wireless communication and power transfer. The implantable medical device includes a motor and an extension rod operably coupled to the motor and configured to extend and retract in response to operation of the motor. The implantable medical device further includes a receive antenna configured to receive a power signal from the external controller. The external controller wirelessly transmits the power signal with communication data embedded therein to control operation of the implantable medical device and receives feedback signals from the implantable medical device to enable real-time monitoring and control of the therapeutic functions.

### REFERENCE TO RELATED APPLICATION

This application claims priority of U.S. provisional patent application Ser. No. 63/691,828, entitled Implantable Medical Device and External Controller for Wirelessly Powering the Implantable Medical Device, filed Sep. 6, 2024, and hereby incorporates this patent application by reference herein in its entirety.

### TECHNICAL FIELD

The apparatus and methods described below generally relate to wirelessly powered implantable medical devices and external controllers for non-invasive therapeutic treatment. The system includes an implantable medical device with a motor-driven extension mechanism that can be wirelessly powered and controlled by an external controller through electromagnetic induction and bidirectional communication protocols.

### BACKGROUND

Scoliosis is a medical condition characterized by an abnormal lateral curvature of the spine. Early onset, progressive scoliosis, if left untreated, can lead to significant deformity, respiratory issues, and reduced quality of life. Traditional treatment methods oftentimes involved invasive surgeries with potential complications and limited flexibility for growth accommodation.

Implantable growing rods represent a significant advancement in the treatment of early onset scoliosis. These implantable devices are designed to provide continuous correction and maintenance of spinal curvature while allowing for natural growth of the spine. The system typically consists of extensible rods anchored to the spine at strategic locations above and below the curve.

The key innovation of motorized growing rods lies in their ability to be lengthened non-invasively using an external controller. This controller, operated by a medical professional, can adjust the length of the rods periodically to match the patient's growth and to optimize curve correction. The external control mechanism eliminates the need for repeated surgical interventions, which were necessary with traditional growing rod systems.

The motorized mechanism within the rods may employ various technologies, such as magnetic actuation or miniature electric motors, to achieve precise and controlled lengthening. This approach allows for more frequent, gradual adjustments, potentially leading to improved outcomes and reduced risk of complications associated with sudden, large corrections.

By combining the benefits of continuous correction with non-invasive adjustment capabilities, motorized growing rods offer a promising solution for managing progressive scoliosis, especially in early onset scoliosis, potentially improving treatment efficacy and patient quality of life.

### BRIEF DESCRIPTION OF THE DRAWINGS

Various embodiments will become better understood with regard to the following description, appended claims and accompanying drawings wherein:

FIG. 1 is an isometric view depicting a system that includes an external controller and a growing rod, in accordance with one embodiment;

FIG. 2 is a cross sectional view of the growing rod of FIG. 1 with an extension rod shown in a retracted position;

FIG. 3 is a cross sectional view of the growing rod of FIG. 1 with an extension rod shown in an extended position;

FIG. 4 is an enlarged view of the growing rod of FIG. 2;

FIG. 5 is an isometric view of a portion of a main body of the growing rod of FIG. 1;

FIG. 6 is a sectional view of a portion of the main body of the growing rod of FIG. 1;

FIG. 7 is an isometric, partially transparent view of the growing rod of FIG. 1;

FIG. 8 is a partially exploded isometric view depicting a dual purpose module of the growing rod of FIG. 1;

FIG. 9 is an enlarged view of the growing rod of FIG. 1 with certain components of a main body removed for clarity of illustration;

FIG. 10 is a schematic view of an electronics package of the growing rod of FIG. 1;

FIG. 11 is a cross sectional view of the growing rod of FIG. 1 with the extension rod shown in a sterilizing position;

FIG. 12 is an exploded isometric view of the external controller of FIG. 1;

FIG. 13 is a partially exploded isometric view of a dual antenna assembly of the external controller of FIG. 12;

FIG. 14 is a schematic view of an electronics package of the external controller of FIG. 1;

FIG. 15 depicts a schematic view of the system of FIG. 1 in association with a remote computing environment; and

FIG. 16 is a cross sectional view of a growing rod in accordance with another embodiment.

### DETAILED DESCRIPTION

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Described herein are example embodiments of a growing rod that can be implanted in a patient and attached to their spine to facilitate correction of scoliosis or other spinal deformities. Once the growing rod is implanted in a patient, it is embedded beneath the patient's skin and soft tissue, making it physically inaccessible without surgical intervention. The growing rod can be a passively powered device that is devoid of an onboard power storage device, such as a battery, for independently powering a motor and any other electrical components onboard the growing rod that are responsible for facilitating operation of the motor to extend and retract the extension rod. As such, an external controller can be used to wirelessly, and non-invasively, power and communicate with the growing rod from outside of the patient's body to control the length of the growing rod (i.e., through extension/retraction of an extension rod) as part of a treatment regimen.

In the illustrated embodiments, although a growing rod is shown and described, it is to be appreciated that the principles and features described herein can be employed in any implantable medical device that can be powered with an external controller through a patient''s skin. Such alternative medical devices may include external fixator struts for bone lengthening and fracture repair, linear actuators for various orthopedic applications, telescoping distractor devices for cranial or facial reconstruction, left ventricular assist devices for cardiac support, implantable drug delivery pumps, neurostimulation devices, cochlear implants, and other therapeutic devices requiring wireless power and control. Additionally, the wireless power transfer and communication principles described herein may be applied to non-medical applications including wirelessly powered sensors, actuators, and control systems in industrial, automotive, aerospace, or consumer electronics applications where remote power delivery and bidirectional communication are beneficial.

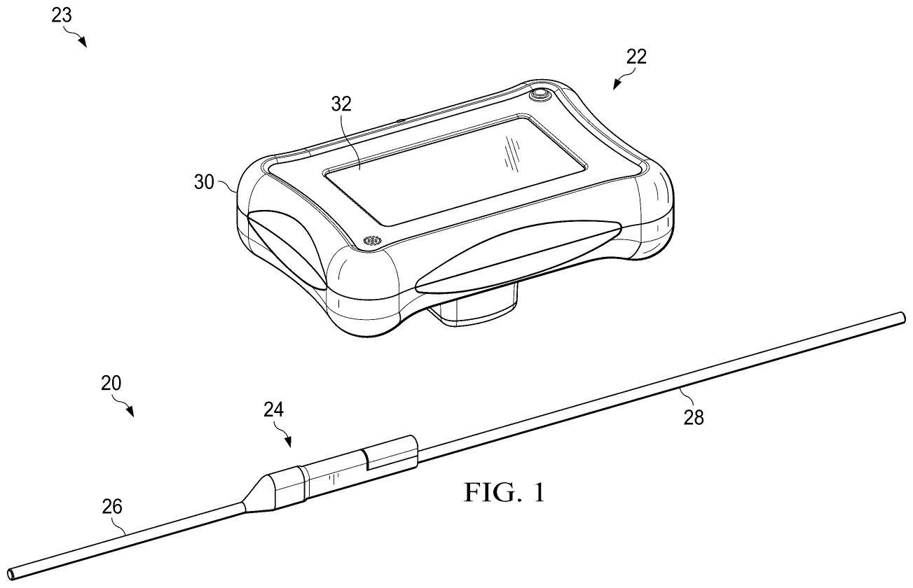

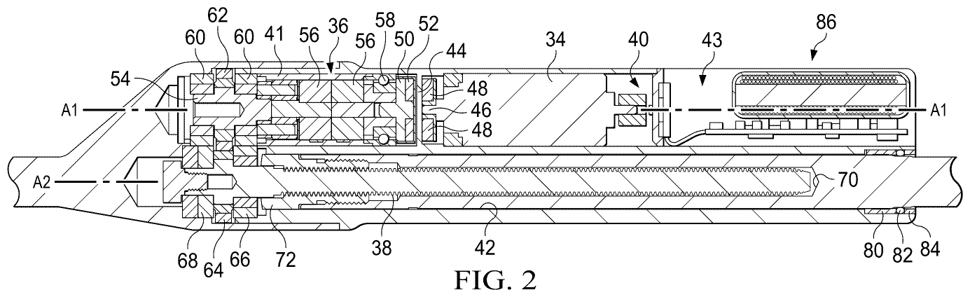

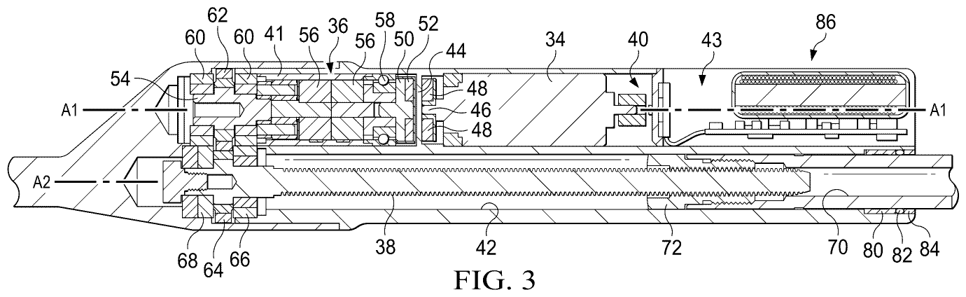

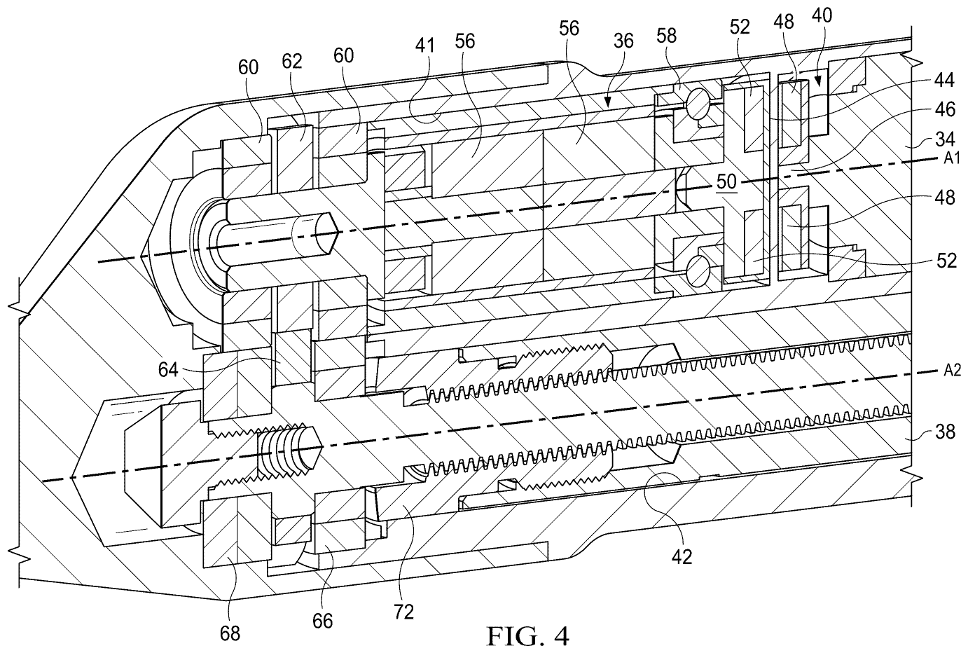

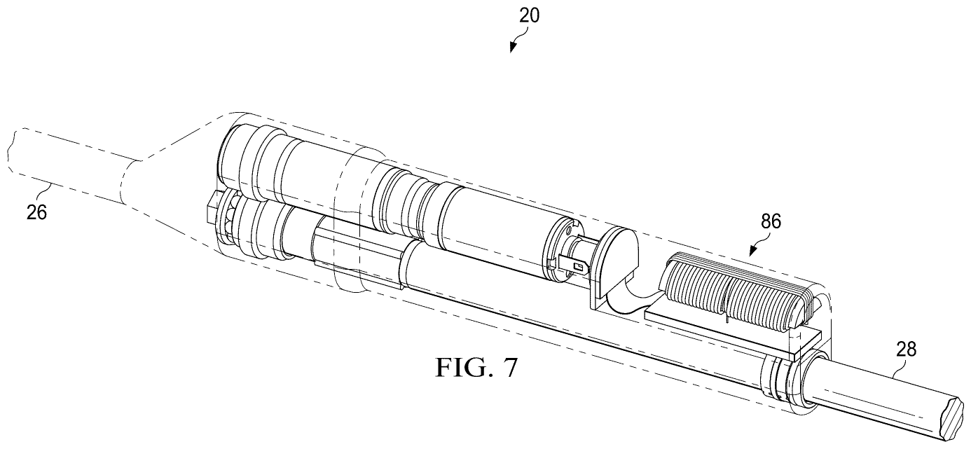

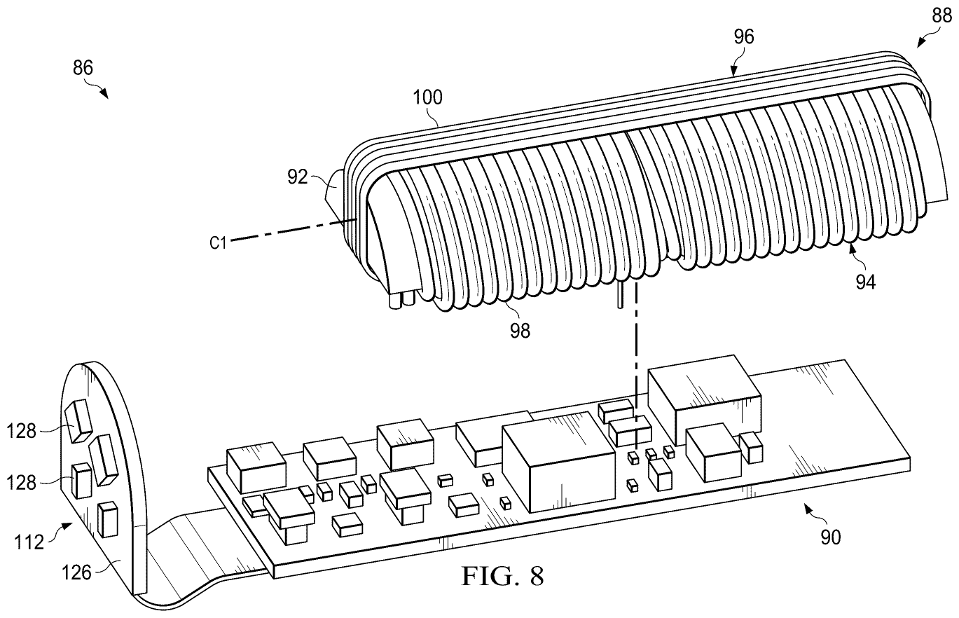

Embodiments are hereinafter described in detail in connection with the views and examples of FIGS. 1-16, wherein like numbers indicate the same or corresponding elements throughout the views. FIG. 1 depicts a growing rod 20 and an external controller 22 (collectively “the system 23”) for use in the treatment of scoliosis. The growing rod 20 can include a main body 24 that includes a rigid rod 26 extending from one side of the main body 24 and an extension rod 28 that is slidably coupled with the main body 24 and that extends from the other side of the main body 24. The extension rod 28 can be slidable with respect to the main body 24 between a retracted position and an extended position. The rigid rod 26 can define an internal cavity or channel that accommodates at least a portion of the extension rod 28, allowing the extension rod 28 to slide telescopically within the rigid rod 26. A motor (34 in FIGS. 2 and 3) can be disposed in the main body 24 and operably coupled with the extension rod 28 to facilitate motorized extension and retraction of the extension rod 28.

The growing rod 20 can be implanted on a patient's spine by attaching the rigid rod 26 and the extension rod 28 at different locations on the patient's spine such that sliding of the extension rod 28 (via the motor 34) can impart a distraction force on the patient's spine to facilitate straightening of the patient's spine to correct any abnormal curvature. The rigid and extension rods 26, 28 can include any of a variety of attachment features (not shown) that facilitate the attachment of the growing rod 20 to a spine, such as, for example, apertures (for receiving a pedicle screw), hooks, clamps, connectors for coupling to existing spinal hardware, laminar hooks that engage the lamina of vertebrae, transverse process hooks that attach to the transverse processes, sublaminar wires or cables that pass beneath the lamina, or rod-to-rod connectors that may allow attachment to previously implanted spinal rods. In some cases, the attachment features can include polyaxial connectors that allow for angular adjustment during implantation, or modular connection systems that permit intraoperative customization of the attachment configuration. The main body 24 and the extension rod 28 can be formed of titanium, stainless steel, or any of a variety of suitable corrosion resistant materials or combinations thereof that are suitable for use in an implantable medical device.

Still referring to FIG. 1, the external controller 22 can include a housing 30 and a display 32 that provides a user interface through which a user (e.g., a medical practitioner) can interact with the external controller 22. An electronics package (e.g., 102 in FIG. 10 and discussed below) can be housed within the housing 30 for wirelessly interacting with the growing rod 20 in the form of a power signal for powering the growing rod 20 and communication data for communicating with the growing rod 20. When a user wants to use the external controller 22 to interact with the growing rod 20, the external controller 22 can be moved proximate enough to the growing rod 20 to facilitate electrical and communicative coupling therebetween. Once that link is established, the external controller 22 can wirelessly power the growing rod 20 and while simultaneously transmitting control data to the growing rod 20. The growing rod 20 can respond by transmitting a feedback signal back to the external controller 22. The proximity required for effective operation can depend on various factors including the power output of the external controller 22, the sensitivity of the receiving components of the growing rod 20, and the electromagnetic properties of the intervening tissue. In some cases, the external controller 22 may need to be positioned within a few centimeters of the implanted growing rod 20 to achieve adequate power transfer and reliable communication. The feedback signal can include feedback data that provides relevant information to the external controller 22 for enabling real-time monitoring and control of the operation of the growing rod 20. Each of the power signal and the feedback signal can be a radio frequency (“RF”) signal which can be understood to mean that they comprise alternating electromagnetic fields that can transmit both energy and information wirelessly across distances. These signals can penetrate biological tissue, making them particularly suitable for communicating with and powering implanted medical devices without requiring direct physical contact or surgical access.

The power signal can be transmitted wirelessly through the patient's skin using electromagnetic induction. In some embodiments, the external controller 22 can generate an alternating electromagnetic field that can penetrate through the patient's skin and soft tissue to reach the implanted growing rod 20. The growing rod 20 can capture this electromagnetic energy and convert it into electrical power for operating the motor 34 and other onboard electrical components. This inductive power transfer approach can allow the growing rod 20 to receive sufficient energy for operation without requiring direct physical contact or surgical access.

In one embodiment, the power signal can be modulated to carry communication data, effectively serving as a carrier signal for both power delivery and data transmission. When the modulated power signal reaches the growing rod 20, the growing rod 20 can extract both the power and the embedded communication data from the same signal. This dual-purpose approach can provide an efficient form of wireless communication that eliminates the need for separate power and data transmission channels, thereby simplifying the wireless interface between the external controller 22 and the implanted growing rod 20.

As such, the feedback signal can be transmitted from the growing rod 20 to the external controller 22 using a separate communication channel that operates independently of the power signal transmission. In some instances, the growing rod 20 can generate and broadcast the feedback signal at a frequency that differs from the power signal carrier frequency. This frequency separation can help minimize interference between the power delivery and data feedback channels, allowing for simultaneous bidirectional communication. Additional details of the power signal (e.g., modulated power signal) and the feedback signal are provided below.

The communication between the growing rod 20 and the external controller 22 can accordingly establish a closed feedback loop that enables precise control and monitoring of the operation of the growing rod 20. In this feedback loop, the external controller 22 can transmit control commands via the control data, that instruct the growing rod 20 to perform specific lengthening operations such as, for example, extending the extension rod 28 to a specific length/position, retracting the extension rod 28 to a specific length/position, applying a particular distraction force, setting a specified power for the motor, achieving or adjusting the extension speed, monitoring real-time force feedback during extension, and verifying the final position of the extension rod 28 after adjustment.

As the growing rod 20 executes these lengthening operations, it can continuously monitor various operational parameters through various local sensors (including environmental sensors, such as, temperature and humidity sensors) and components, motor current measurements, and/or voltages. The growing rod 20 can then transmit real-time or near real-time feedback data about these operational parameters back to the external controller 22 through the separate feedback signal channel. The external controller 22 can process this feedback data and can automatically adjust the operation of the growing rod 20 via control commands based on the received information, creating a dynamic control system that can respond to changing conditions during the lengthening procedure.

The external controller 22 can also be configured to detect fault conditions present on the growing rod 20 based on the feedback data received from the implanted device, and can automatically terminate operation while providing appropriate notices to the user through the display 32 or other alert mechanisms. In some aspects, the external controller 22 can implement diagnostic algorithms that continuously analyze the feedback data to identify abnormal operating conditions that could indicate device malfunction or potential safety concerns. Some examples of the fault conditions that can be detected include motor overcurrent conditions indicating excessive resistance or mechanical binding, internal temperature readings exceeding safe operating thresholds, excessive humidity, loss of communication signal integrity or complete signal dropout, motor position encoder failures or inconsistent position feedback, power reception efficiency below acceptable levels indicating poor coupling or antenna malfunction, unexpected motor stall conditions during extension or retraction operations, force measurements exceeding predetermined safety limits, memory corruption or data integrity errors in the growing rod 20, mechanical component wear indicators suggesting device degradation, electromagnetic interference levels that compromise reliable operation, and timeout conditions where commanded operations fail to complete within expected timeframes.

For purposes of illustration, various examples of the response of the external controller 22 to certain feedback data during a lengthening operation will now be described. In one example, the external controller 22 can respond to motor current measurement feedback data by automatically adjusting the power delivery when the feedback data indicates that the motor current has increased beyond a predetermined threshold. For example, if the current draw of the motor 34 exceeds normal operating parameters due to increased resistance from spinal stiffness, the external controller 22 may reduce the extension speed or temporarily pause the lengthening operation (e.g., by slowing or stopping the motor 34) to prevent excessive force application. Conversely, if the current draw of the motor 34 is lower than expected, the external controller 22 may increase the extension rate by increasing the speed of the motor 34 to optimize treatment efficiency while maintaining safe operating conditions.

The external controller 22 can also respond to temperature feedback data by implementing thermal protection protocols. When the feedback data indicates that the internal temperature of the growing rod 20 is approaching upper safety limits, the external controller 22 can automatically reduce power output, decrease motor operation speed, or initiate cooling periods inter or intra the extension cycles. In some instances, if the measured temperature exceed critical thresholds, the system can terminate the lengthening procedure entirely and alert the medical practitioner through visual or audible warnings on the display 32.

In another example, the external controller 22 can respond to motor position feedback data by providing real-time position correction and verification. In such an example, the position of the extension rod 28 can be a function of the rotational position of the motor 34. If the feedback data indicates that the motor 34 has not rotated sufficiently to achieve the desired extension rod position, the external controller 22 can automatically compensate by adjusting motor control parameters, increasing torque output, or implementing alternative extension strategies. The external controller 22 can also detect rely on the motor position feedback data to detect unexpected position changes that could indicate mechanical binding or component malfunction. In these instances, the external controller 22 can attempt to restore proper operation by slightly retracting the extension rod 28 and then attempting to extend it again to its desired position. If the external controller 22 is unable to restore proper operation, it can alert the medical practitioner of the rod failure through visual or audible warnings on the display 32.

In yet another example, the external controller 22 can respond to force feedback measurements by implementing adaptive force limiting protocols. When the feedback data indicates that the distraction force is approaching maximum safe levels before reaching the target extension distance, the system may automatically reduce the extension speed, apply intermittent force pulses, or modify the extension trajectory to achieve gradual tissue adaptation. In some aspects, the system may implement force ramping protocols that gradually increase distraction force over time rather than applying sudden force changes.

The feedback loop established between the external controller 22 and the growing rod 20 can also be utilized for various alternative operational modes other than the lengthening operational mode described above. In one example of an alternative operational mode, the external controller 22 can operate in an interrogation mode where the external controller 22 transmits interrogation commands to the growing rod 20 via the communication data, and the growing rod 20 responds with requested information via the feedback signal. This interrogation mode may allow the external controller 22 to query the growing rod 20 for various types of stored data, operational parameters, and device status information without initiating any therapeutic adjustments or mechanical operations.

In one application, the interrogation mode can be employed to enable the external controller 22 to control only one growing rod when there are more than one growing rod implanted on a patient's spine. When two or more growing rods 20 are installed on a patients spine, typically on opposite sides of the spine, each growing rod can be assigned a unique ID that is stored in memory on the growing rod 20 and may also be provided on a device label for logging and record-keeping purposes. This unique ID can be input by broadcasting it from the growing rod 20 and receiving it into the external controller 22 or alternatively by entering or selecting the unique ID by the user to specifically identify which growing rod 20 the user intends to adjust during a treatment session. In some cases, the unique ID may be displayed as a barcode, QR code, or alphanumeric string on the device packaging or accompanying documentation, allowing medical practitioners to easily reference and enter the correct identifier into the external controller 22 before initiating communication with the implanted device.

When the external controller 22 is positioned over a patient's spine, the external controller 22 can broadcast communication data containing an identification query for device identification to the nearby growing rods. The broadcast can be transmitted at a power level sufficient to reach multiple growing rods within the transmission range. Each growing rod that receives this query can respond with feedback data that includes its unique device identifier and may also include additional device information such as manufacturing date, model number, firmware version, and operational status. The external controller 22 can receive these identification responses and compare them against the target device identifier that was previously entered by the user. Once the correct growing rod 20 is identified, the external controller 22 can establish a dedicated communication channel with that specific device using its unique identifier, ensuring that subsequent communication data and power signals are directed only to the intended growing rod 20 while avoiding interference with other implanted devices.

The interrogation mode can additionally or alternatively be utilized in various other applications to obtain different types of information (via the feedback signal) from the growing rod 20. In one instance, the interrogation mode can also be used to obtain real-time diagnostic information such as current power consumption indicators, internal component health assessments, calibration data, sensor readings, and system integrity checks. In other instances, the external controller 22 can query the growing rod 20 for configuration parameters including maximum force thresholds, extension speed limits, safety timeout values, and operational mode settings. These methods provide a system for facilitating avoidance of interference from similar devices.

The external controller 22 can also interrogate the growing rod 20 to retrieve historical operational information including cumulative extension distance records, total operating hours, force application history, temperature exposure logs, and motor performance metrics over time. The external controller 22 can respond to historical operational data by implementing predictive adjustments. When feedback data indicates patterns in previous lengthening sessions, such as consistent resistance at specific extension distances, the system may preemptively adjust power delivery, modify extension speeds, or implement specialized protocols to address anticipated challenges before they occur. The interrogation mode may additionally allow retrieval of error logs, fault condition histories, maintenance schedules, and device performance analytics that can inform clinical decision-making and device management protocols.

In another example of an alternative operational mode, the external controller 22 can operate in a programming mode where the external controller 22 transmits programming commands to the growing rod 20 via the communication data, and the growing rod 20 responds with confirmation data via the feedback signal. This programming mode may allow the external controller 22 to modify operational parameters, update firmware, and configure device settings within the growing rod 20 without requiring physical access to the implanted device.

The growing rod 20 can respond to programming commands by transmitting confirmation data through the feedback signal that verifies successful parameter updates and provides status information about the programming operation. This confirmation data may include verification codes that confirm specific parameters have been successfully updated, error messages indicating any programming failures or conflicts, current parameter values after the update to verify accuracy, memory status indicators showing available storage capacity, and system integrity checks confirming that the programming operation has not compromised device functionality. In some instances, such data confirmation can occur in real-time

In one application, the programming mode can be employed to enable calibration of the growing rod 20. During calibration operations, the external controller 22 can transmit specific calibration commands to the growing rod 20 that instruct the device to perform predetermined mechanical movements or sensor readings while monitoring the corresponding feedback data to establish baseline operational parameters. The calibration process may involve commanding the motor 34 to rotate among specific rotational positions while the positional data of the motor 34 is reported to the external controller 22 via the feedback data, allowing the external controller 22 to verify the accuracy of position measurements and detect any drift or offset errors that may have developed over time. The external controller 22 can also initiate force calibration sequences where the extension rod 28 is moved to known positions against controlled resistance, enabling the system to correlate motor current measurements with actual force output and update internal calibration coefficients accordingly. Temperature calibration can also be performed by monitoring the internal temperature of the growing rod 20 that is reported by the feedback data under various operational conditions and comparing them against expected thermal profiles to ensure accurate temperature monitoring throughout the device's operational range. These calibration sequences can provide a mechanism to measure initial parameter values and adjust power consumption levels during extension of the extension rod 28 to draw more or less current based on optimized values without requiring dedicated load sensors.

The external controller 22 can process the calibration feedback data to adjust or modify internal parameters stored in the growing rod 20, such as position scaling factors, force conversion coefficients, temperature compensation values, motor control parameters, force threshold limits, extension speed parameters, safety timeout values, alarm thresholds, operational mode preferences, communication protocols. These internal parameters can be customized to be patient-specific, taking into account individual factors such as age, weight, spinal condition severity, and treatment goals to optimize device performance for each patient's unique physiological characteristics. In some instances, the calibration process can reveal systematic errors or component degradation that requires adjustment of operational thresholds or safety limits to maintain proper device function. The external controller 22 can generate calibration reports that document the calibration results, including any parameter adjustments made, detected deviations from expected performance, and recommendations for future calibration intervals. This calibration capability may be particularly valuable for ensuring long-term accuracy and reliability of the growing rod 20 throughout its implantation period, as it allows for correction of gradual changes in component characteristics that could otherwise affect treatment precision or safety margins.

In another application, the programming mode can be employed to update firmware or software within the growing rod 20 to enhance device functionality, correct software issues, or add new operational capabilities. The external controller 22 can transmit update packages in segments through the communication data, with each segment being verified and acknowledged by the feedback data before proceeding to the next portion. The growing rod 20 can ultimately respond with confirmation feedback data indicating successful receipt and installation of each firmware segment, progress indicators showing the completion status of the update process, and final verification messages confirming successful firmware installation and system restart.

The programming mode can incorporate security protocols to prevent unauthorized modifications to device parameters. The external controller 22 can include authentication mechanisms that verify the legitimacy of programming commands before transmission, and the growing rod 20 can implement verification procedures that confirm the authenticity of received programming instructions. The confirmation data transmitted by the growing rod 20 can include security status indicators, authentication verification results, and access log information that documents programming activities for audit and safety purposes.

In addition to the lengthening, interrogation, and programming modes described above, the external controller 22 may support various other discrete operational modes that can be selected and executed based on specific clinical requirements or device maintenance needs. One example of a discrete operating mode is a signal quality and alignment mode which can be employed by the external controller 22 to detect and compensate for any signal quality or alignment issues that might occur during communicative and electrical coupling between the growing rod 20 and the external controller 22. In such an example, the signal quality can be monitored by the external controller 22 via the feedback data. When the signal quality degrades, the feedback data can trigger automatic communication optimization responses from the external controller 22 which can include adjusting transmission power levels, modifying carrier frequencies, or altering modulation parameters to maintain reliable communication. The external controller 22 can also provide real-time guidance to the user through the display 32 to optimize device positioning and alignment.

Another example of a discrete operating mode is a power management mode which can be employed by the external controller 22 to control the depletion of its internal battery as a function of the power consumption by the growing rod 20. In such an example, the external controller 22 can respond to power consumption feedback from the growing rod 20 by adjusting operational parameters at the growing rod to reduce power consumption in order to extend treatment session duration. If the feedback data indicates higher than expected power consumption, the external controller 22 can reduce motor speed, implement power-saving modes during idle periods, or optimize the duty cycle of power transmission to maximize the available treatment time.

Another example of a discrete operating mode is a fault response mode. When the external controller 22 detects a fault, as described above, the external controller 22 can transmit emergency stop commands to the growing rod 20 that immediately halts all motor operations. The feedback signal provides immediate confirmation of the emergency stop execution along with current device status information.

It is to be appreciated that any of a variety of suitable alternative operating modes can be supported by the feedback loop established between the external controller 22 and the growing rod 20, such as, for example, diagnostic testing modes, maintenance verification protocols, and specialized therapeutic protocols that may be developed for specific patient conditions or treatment requirements. In these instances, the system can employ the same communication protocols and monitoring capabilities for diagnostic testing, device calibration, parameter adjustment, and maintenance operations that may be performed throughout the implantation period.

It is also to be appreciated that the operational modes described herein may be implemented individually or in combination with one another to provide comprehensive device management capabilities. In some aspects, multiple operational modes can be executed simultaneously, such as operating in lengthening mode while concurrently monitoring signal quality and power consumption through the signal quality and alignment mode and power management mode, respectively. The external controller 22 may automatically transition between different operational modes based on detected conditions or user-defined protocols, such as automatically entering fault response mode when abnormal operational parameters are detected, or switching to power management mode when battery levels fall below predetermined thresholds. Further it should be appreciated that these modes, features, and various control systems all require power and computational capabilities either onboard the growing rod 20 and/or within the external controller 22. It is still further to be appreciated that the efficiency of power transmission given the small volume is a physical constraint which challenges even current size of electronics and electromagnetic principles. The system described can be achieved without on board energy storage and operates only from externally transmitted power.

The activation of operational modes can be achieved through manual user selection via the display 32, where medical practitioners can choose specific modes based on clinical requirements or treatment protocols. In some cases, the external controller 22 may present menu options or interface elements that allow users to select individual modes or predefined mode combinations for specific procedures. Alternatively, operational modes can be automatically activated based on programmed logic, sensor feedback, or predetermined schedules stored within the external controller 22. For example, the system may automatically initiate interrogation mode at regular intervals to collect diagnostic data, or automatically enter calibration mode when specific time intervals have elapsed since the last calibration procedure. The external controller 22 may also implement conditional mode activation, where certain operational modes are triggered automatically when specific feedback data patterns or device conditions are detected, enabling responsive and adaptive device management throughout the treatment period.

The external controller 22 can incorporate comprehensive data logging capabilities that continuously record feedback data received from the growing rod 20 throughout treatment sessions. The logged feedback data can include mechanical resistance measurements, motor current draw patterns, force application profiles, extension speeds, and operational parameters that collectively characterize the biomechanical response of the patient's spine during lengthening procedures. This historical data collection enables the external controller 22 to build a comprehensive profile of the patient's spinal characteristics and treatment response patterns over multiple lengthening sessions. The data logging system may timestamp all recorded parameters and associate them with specific treatment events, extension distances, and patient identifiers to create a detailed treatment history that can be analyzed for patterns and trends.

The external controller 22 can implement analytical algorithms that process the logged feedback data to generate treatment recommendations and parameter adjustments for future lengthening procedures based on the feedback data. For example, if the external controller 22 detect consistent patterns in mechanical resistance at specific extension distances, it can automatically suggest modified extension protocols that anticipate and accommodate the identified resistance patterns. In particular, if the logged data indicates that mechanical resistance consistently increases at particular extension distances across multiple treatment sessions, the external controller 22 may recommend implementing gradual force ramping protocols, extended dwell times at resistance points, or modified extension speeds to optimize patient comfort and treatment efficacy. The system may also identify trends in the patient's biomechanical response over time, such as decreasing resistance patterns that may indicate successful spinal adaptation, or increasing resistance that may suggest the need for alternative treatment approaches.

The analytical capabilities may extend to predictive modeling where the external controller 22 uses machine learning algorithms or statistical analysis methods to forecast optimal treatment parameters for upcoming sessions based on historical feedback patterns. The external controller 22 can generate recommendations for extension distances, force application profiles, treatment session timing, and safety thresholds that are customized to the individual patient's biomechanical characteristics as revealed through the accumulated feedback data. These predictive recommendations may be presented to medical practitioners through the display 32, enabling evidence-based treatment planning that incorporates the patient's specific response history and biomechanical profile. In some instances, the analytical processing and recommendation generation may be performed partially or entirely on remote computing devices, where more sophisticated computational resources and advanced algorithms can be employed to analyze the collected data and generate treatment recommendations that are subsequently transmitted back to the external controller 22 or a separate display for presentation to the medical practitioner.

The logged feedback data collected by the external controller 22 can also be utilized to enable self-calibration capabilities for the growing rod 20, allowing the device to automatically adjust its operational parameters based on accumulated performance data over time. The self-calibration process can be implemented by the external controller 22 and/or a remote computing device to analyze historical motor current measurements, position encoder readings, force application data, and temperature profiles to identify systematic deviations from expected performance characteristics. By comparing actual operational parameters against theoretical or baseline values, the system can detect gradual changes in component behavior that may result from normal wear, environmental factors, or aging of mechanical and electrical components within the implanted device.

The self-calibration algorithms can process the logged data to identify trends and patterns that indicate the need for parameter adjustments. For example, if the historical data shows that motor current draw has gradually increased over multiple treatment sessions to achieve the same extension force, the system may automatically adjust motor control parameters to compensate for increased mechanical resistance or motor efficiency changes. Similarly, if motor position readings show consistent offset errors when compared to expected extension distances, the calibration system can automatically update position scaling factors or implement correction algorithms to maintain accurate length measurements. The self-calibration process may also analyze temperature data patterns to adjust thermal compensation parameters, ensuring that temperature variations do not adversely affect device performance or measurement accuracy.

The implementation of self-calibration capabilities may involve the external controller 22 transmitting updated calibration parameters to the growing rod 20 through the programming mode described above. The growing rod 20 can store these updated parameters in its internal memory and apply them during subsequent operations to improve accuracy and performance. The self-calibration process can be executed automatically at predetermined intervals, such as after a specific number of treatment sessions or when accumulated data reaches sufficient statistical significance to enable reliable parameter adjustments. In some cases, the self-calibration algorithms can incorporate machine learning techniques that continuously refine calibration parameters based on ongoing data collection, enabling the system to adapt to changing conditions and maintain optimal performance throughout the extended implantation period. The self-calibration results may be documented in calibration logs that can be reviewed by medical practitioners to verify system performance and identify any trends that may require clinical attention or device maintenance.

Referring now to FIGS. 2 and 3, the growing rod 20 can include a motor 34, a gear box 36, and a lead screw 38 (collectively “the drive components”) that are operably coupled together and that cooperate to facilitate sliding of the extension rod 28 with respect to the main body 24 between a retracted position (FIG. 2) and an extended position (FIG. 3). The main body 24 can define a motor compartment 40 that houses the motor 34, a gear box compartment 41 that houses the gear box 36, and a lower compartment 42 that accommodates the lead screw 38 The extension rod 28 can be at least partially disposed in the lower compartment 42 and can extend distally therefrom. The motor and gear box compartments 40, 41 can be longitudinally adjacent to one another (i.e., positioned vertically relative to each other) but can be physically isolated from each other, as will be described in further detail below. The motor and gear box compartments 40, 41 can be separated by a wall 44, and the motor 34 and the gear box 36 can be located on opposite sides of the wall 44. The gear box and lower compartments 41, 42 can be laterally adjacent to one another (i.e., positioned horizontally relative to each other) and contiguous such that they are in fluid communication with each other.

Referring now to FIGS. 2-4, the motor 34 can include an output shaft 46 that is rotatable when the motor 34 is activated. The output shaft 46 can include a plurality of magnets 48 that are radially spaced from each other and positioned adjacent to the wall 44. The gear box 36 can include an input shaft 50 that includes a plurality of magnets 52 that are radially spaced from each other and positioned adjacent to the wall 44 on an opposite side of the magnets 48. The magnets 48, 52 can magnetically couple the output shaft 46 and the input shaft 50 together through the wall 44 such that rotation of the output shaft 46 can correspondingly rotate the input shaft 50 of the gear box 36 even though they are not mechanically coupled together.

The gear box 36 can include an output shaft 54 that is operably coupled with the input shaft 50 by a plurality of reduction stages 56 that define a reductive gear ratio between the input shaft 50 and the output shaft 54. In one embodiment, the gear ratio can be about 1466:1. The input shaft 50 and the output shaft 54 can be journalled with respect to the main body 24 by respective bearings 58, 60. A drive gear 62 can be coupled with the output shaft 54. The output shaft 46, the input shaft 50, the output shaft 54, and the drive gear 62 can rotate coaxially about a first axis A1.

A driven gear 64 can be coupled with the lead screw 38 and intermeshed with the drive gear 62 such that rotation of the output shaft 54 causes rotation of the lead screw 38 in an opposite direction as the output shaft 54. The gears 64 can be formed of a thermoplastic, such as PEEK, or any of a variety of suitable alternative biocompatible materials. The lead screw 38 can be journalled with respect to the main body 24 by a pair of bearings 66, 68. The lead screw 38 and the driven gear 64 can rotate coaxially about a second axis A2 that is substantially parallel with the first axis A1 but axially offset from the first axis A1. The lead screw 38 can be threadedly coupled with the extension rod 28 such that rotation of the lead screw 38 causes the extension rod 28 to slide (e.g., translate) between the retracted position (FIG. 2) and the extended position (FIG. 3) along the second axis A2. While the lead screw 38 is described as being threadedly coupled with the extension rod 28, it is to be appreciated that any of a variety of translatable coupling arrangements may be employed to convert rotational motion of the lead screw 38 into linear translation of the extension rod 28. Alternative coupling mechanisms may include ball screw assemblies, rack and pinion systems, cam-follower arrangements, or other mechanical linkages that facilitate controlled extension and retraction of the extension rod 28 in response to rotation of the lead screw 38.

Providing the motor 34 and the gear box 36 on a different axis as the lead screw 38 (e.g., with the lead screw 38 located beneath the motor 34 and the gear box 36) allows the drive path between the motor 34 and the extension rod 28 to effectively make a U-turn (e.g., at the drive and driven gears 62, 64) which can result in a more compact form factor than conventional single axis arrangements (e.g., where the drive components are positioned along the same axis) and can thus be more suitable for when anatomical constraints are present on a patient (e.g., when used in a pediatric patient). This form factor can also provide a greater surface area which allows for overlying tissue to be spread over a wider surface area thereby reducing pressure on the skin. This can be helpful to patients having limited soft tissue coverage over the implant, such as pediatric patients with lower BMI, which is oftentimes common in early onset scoliosis.

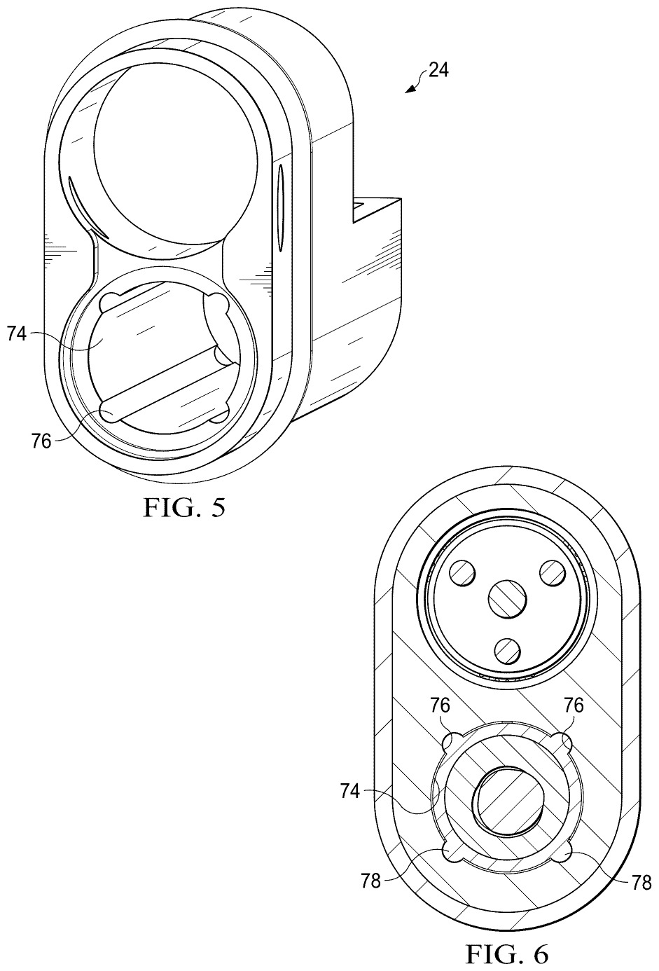

Still referring to FIGS. 2-4, the extension rod 28 can define an inner bore 70 that accommodates the lead screw 38. A nut 72 can be secured to a proximal end of the extension rod 28 and can be threadedly engaged with external threads of the lead screw 38 such that the nut 72 is effectively responsible for sliding the extension rod 28 as a function of the rotation of the lead screw 38. The nut 72 can be formed of a thermoplastic, such as PEEK, or any of a variety of suitable alternative biocompatible materials. In an alternative embodiment, the inner bore 70 might be internally threaded to interface directly with the lead screw 38 without use of the nut 72. As illustrated in FIGS. 5 and 6, a portion of the main body 24 is shown to include an interior wall 74 that at least partially defines the third compartment 42 and includes a plurality of grooves 76 that extend longitudinally along the third compartment 42. The nut 72 can include a plurality of splines 78 that extend into the grooves 76 to prevent the nut 72 from rotating while also allowing the nut 72 to slide longitudinally along the interior wall 74 together with the extension rod 28.

When the extension rod 28 is to be extended, the motor 34 can be activated to rotate the output shaft 54 about the first axis A1 in a first direction (e.g., clockwise) which in turn causes the input shaft 50 of the gear box 36 to rotate in the same direction due to the magnetic interaction between the magnets 48, 52. In response, the output shaft 54 of the gear box 36 and the drive gear 62 can rotate in the first direction but at a lower speed and a higher torque than the input shaft 50. The speed and torque of the output shaft 54 can be governed by the gear ratio of the gear box 36. The rotation of the drive gear 62 can cause the driven gear 64 and the lead screw 38 to rotate about the second axis A2 but in an opposite direction as the drive gear 62 (e.g., counterclockwise). The lead screw 38 can be rotated in an unthreading direction which can cause the extension rod 28 to elongate along the axis A2. When the extension rod 28 is to be retracted, the operation of the motor 34 can be reversed which can reverse the rotation of the output shaft 54 (e.g., in a counterclockwise direction) thus causing the lead screw to rotate in a threading direction to retract the extension rod 28. In some embodiments, the motor 34 can be a DC motor such that reversing the operation of the motor 34 can be achieved by reversing the polarity of the DC voltage across the motor 34.

When the extension rod 28 is lengthened, the growing rod 20 imparts a distraction force onto the patient's spine that helps with straightening of the spine. The distraction force that is necessary to straighten the spine during lengthening of the extension rod 28 can increase as the extension rod 28 extends towards the desired length in order to overcome the spine's inherent physiological resistance to being straightened. In addition, the distraction force that is required to straighten the spine can be greater at each subsequent patient visit as the spine becomes straighter. The distraction force imparted by the growing rod 28 can be a function of the magnitude of current that is applied to the motor 34 and can be substantially proportional to the current (e.g., an increase in current increases the speed of the motor 34 to increase the distraction force). Therefore, as the need for additional distraction force increases, either during lengthening or between lengthening sessions, the current applied to the motor 34 can be increased to change the distraction force accordingly.

Excessive distraction forces on the spine, however, can increase the risk of causing harm to the patient, such as, for example, damage to the spinal structure or surrounding tissue (e.g., nerves and blood vessels), pain or discomfort, and/or dislodging of the growing rod 20 from the patient's spine. As mentioned above, the current applied to the motor 34 can be controlled to prevent the distraction force from reaching a harmful level. More particularly, a predefined limit can be set for the current of the motor 34 that correlates to a maximum acceptable distraction force of the growing rod 20. During operation of the motor 34, the current that is applied to the motor 34 can be maintained at or below the predefined limit to prevent the distraction force from exceeding the acceptable threshold throughout the extension of the extension rod 28 to the desired length. If the current imparted to the motor 34 is consistently at the threshold and the extension rod 28 has stalled, the operation of the motor 34 can be terminated and a notification can be provided to the user on the display 32 or via another source, such as an audible alarm, to indicate that the distraction force required to lengthen the growing rod 20 is too high and requires attention.

As illustrated in FIGS. 2 and 3, a bushing 80 can be provided at a distal end of the third compartment 42 between the interior wall 74 and the extension rod 28. The bushing 80 can guide and support the sliding of the extension rod 28 with respect to the main body 24. The bushing 80 can be formed of a thermoplastic, such as PEEK, or any of a variety of suitable alternative low-friction biocompatible materials. An O-ring 82 can be provided distally adjacent to the bushing 80 and can cooperate with the bushing 80 to provide effective sealing of the third compartment 42 from the surrounding environment. The bushing 80 and the O-ring 82 can be held in place by a retention cap 84. In some instances, the resulting seal between the bushing 80, the O-ring 82, and the extension rod 28 might not be completely impervious to fluid and thus might still allow the gear box compartment 41 and the lower compartment 42 to be exposed to the surrounding environment such that trace amounts of environmental contaminants from the surrounding environment, such as bodily fluids and/or other foreign substances, are able to infiltrate the lower compartment 42 or contaminates from the lower compartment 42 are able to be introduced into the surrounding environment, especially during sliding of the extension rod 28. As a result, the gear box compartment 41 and the lower compartment 42 can be considered to be a “wet zone.” The gear box 36, the lead screw 38, and the associated components can accordingly be wet-rated and thus configured to withstand any environmental contaminants that might inadvertently be introduced into the gear box compartment 41 and the lower compartment 42. While fluid ingress and egress may be unavoidable to and from the wet zone, in the event the drive components produce any wear debris in the gear box compartment 41 and the lower compartment 42, the bushing 80 and the O-ring 82 can be configured to prevent the wear debris from exiting the growing rod 20 and reaching the patient tissue. While the bushing 80 and O-ring 82 represent one example of a sealing arrangement, it is to be appreciated that various alternative sealing arrangements may be employed to achieve similar sealing functionality between the extension rod 28 and the main body 24.

During extension of the extension rod 28, the telescoping action of the extension rod 28 sliding outward from the lower compartment 42 may create a negative pressure condition within the lower compartment 42. As the extension rod 28 moves distally and extends beyond the main body 24, the volume within the lower compartment 42 increases while the amount of air or fluid contained therein remains relatively constant. This expansion can result in a pressure differential where the pressure inside the lower compartment 42 becomes lower than the ambient pressure in the surrounding biological environment. The negative pressure condition can act as a barrier that resists the infiltration of bodily fluids, contaminants, or other foreign substances into the third compartment 42 from the external environment.

The negative pressure effect may be particularly pronounced when the extension rod 28 functions as a telescoping plunger within the lower compartment 42. In some cases, this vacuum-like condition can provide an additional protective mechanism that complements the sealing function of the bushing 80 and the O-ring 82. The pressure differential may help to draw any potential contaminants away from the interface between the extension rod 28 and the sealing components, thereby reducing the likelihood of environmental infiltration into the wet zone. This negative pressure condition may also assist in maintaining the integrity of the lower compartment 42 by creating an inward force that can help retain any wear debris or particles that might be generated within the compartment during operation of the mechanical components.

Referring now to FIGS. 2-3 and 7, the growing rod 20 can include a dual-purpose module 86 that is configured to receive the power signal from the external controller 22 and to facilitate communication of the feedback signal to the external controller 22. The dual-purpose module 86 can be disposed in a electronics compartment 43 defined by the main body adjacent to the motor compartment 40. Because the motor 34 and the dual-purpose module 86 are comprised of electrical components, any infiltration of environmental contaminants into the motor compartment 40 or the electronics compartment 43 can be harmful, and in some cases can lead to premature failure of the motor 34 and/or the dual-purpose module 86. As a result, the motor compartment 40 can be hermetically sealed (e.g., during manufacturing) to prevent the infiltration of such environmental contaminants. Because the motor compartment 40 is physically isolated from the gear box compartment 41 and the lower compartment 42 (e.g., by the wall 44) and hermetically sealed, any environmental contaminants that reach the gear box compartment 41 or lower compartment 42 are prevented from being able to infiltrate the motor compartment 40. The dual-purpose module 86 can also be embedded into a potting compound to further enhance protection from environmental contaminants.

As illustrated in FIG. 8, the dual-purpose module 86 can include a dual antenna assembly 88 that is mounted on a printed circuit board (PCB) 90. The dual antenna assembly 88 can include a ferrous core 92, a receive antenna 94 for receiving the power signal from the external controller 22, and a transmit antenna 96 for transmitting the feedback signal that includes feedback data to the external controller 22. The receive antenna 94 can be configured to receive the modulated power signal from the external controller 22. The power delivered from the external controller 22 can be sufficient to power the growing rod 20 such that the growing rod 20 does require an onboard battery or other power storage device which can result in a more compact form factor than conventional growing rods. The transmit antenna 96 can be configured to communicate the feedback signal to the external controller 22. The receive antenna 94 and the transmit antenna 96 can be configured to receive and transmit signals simultaneously when interacting with the external controller 22.

The receive antenna 94 can include windings 98 that are wound circumferentially around the ferrous core 92 such that the windings 98 are substantially coaxial with a centerline C1 that extends longitudinally through the ferrous core 92. This design allows for the growing rod 20 to be rotated 180 degrees about its centerline axis to accommodate both left and right sides of the spine eliminating the needs for specific inventory for left or right rods. The transmit antenna 96 can include windings 100 that are wound longitudinally around the ferrous core 92 and over the receive antenna 94 such that at least a portion of the receive antenna 94 is sandwiched between the transmit antenna 96 and the ferrous core 92. The receive and transmit antennas 94, 96 can therefore utilize the same core (e.g., the ferrous core 92) which provide a significantly more compact antenna design than conventional antenna designs that utilize dedicated ferrous cores for different antennas.

The windings 98 of the receive antenna 94 and the windings 100 of the transmit antenna 96 can be substantially orthogonal to each other which can alleviate cross-talk, interference, malalignment sensitivity (e.g., translation and rotation), and other drawbacks that are oftentimes associated with simultaneously operating antennas that are in close proximity with one another. The orthogonal configuration can create distinct electromagnetic field patterns that operate independently despite sharing the same ferrous core 92. In some instances, the circumferential windings 98 of the receive antenna 94 can generate a magnetic field that is oriented primarily in a radial direction relative to the centerline C1, while the longitudinal windings 100 of the transmit antenna 96 can produce a magnetic field that is oriented primarily in an axial direction along the centerline C1.

This orthogonal field orientation can allow the receive antenna 94 to efficiently couple with the power signal transmitted from the external controller 22 without significantly interfering with the feedback signal being transmitted by the transmit antenna 96. The spatial separation of the electromagnetic field patterns can enable the dual antenna assembly 88 to maintain signal integrity for both power reception and data transmission functions simultaneously. In some cases, the orthogonal winding arrangement may provide electromagnetic isolation between the two antenna functions, allowing the receive antenna 94 to extract power from the incoming signal while the transmit antenna 96 concurrently broadcasts the feedback signal at a different frequency or using a different modulation scheme.

The shared ferrous core 92 can serve as a common magnetic flux concentrator for both antenna functions while the orthogonal winding orientations can ensure that the magnetic flux paths for power reception and feedback transmission remain substantially independent. This configuration can allow for efficient utilization of the available core material while maintaining the distinct operational characteristics required for each antenna function. In some embodiments, the ferrous core 92 can be designed with specific permeability characteristics that optimize performance for both the power reception frequency and the feedback transmission frequency.

The windings 98, 100 can be formed of a coated magnetic wire such as, for example, thermoplastic coated copper wire. Because the receive antenna 94 is responsible for receiving power from the external controller 22, the wire used for the windings 98 can have a thicker gauge than the wire used for the windings 100 of the transmit antenna 96 which is only responsible for transmitting a data signal (e.g., the feedback signal) back to the external controller 22. Using thinner wire for the windings 100 can allow for a greater number of turns to be incorporated into the allotted space for the transmit antenna 96 which can enhance the transmissivity of the feedback signal and can strengthen the electromagnetic field which can result in improved communication with the external controller 22.

In an alternative embodiment, the electronics components including the PCB 90, dual-purpose module 86, and/or associated electrical components can be housed in an additional compartment that is separately affixed to the main body 24 rather than being integrated within the main body structure. This additional compartment can be mechanically coupled to the main body 24 through various attachment mechanisms including threaded connections, welded joints, or interlocking features that provide secure mechanical coupling while maintaining the hermetic integrity of both the additional compartment and the existing compartments within the main body 24. The additional compartment can be positioned at various locations relative to the main body 24, such as adjacent to the motor compartment 40 or extending laterally from the main body structure, depending on anatomical constraints and surgical placement requirements.

The electrical connection between the additional compartment and the motor compartment 40 can be achieved through feedthrough connections that pass through one or more of the existing compartments within the main body 24. These feedthrough connections can utilize hermetically sealed electrical terminals similar to the terminals 134 described above, allowing electrical signals and power to be transmitted between the additional compartment and the motor 34 while maintaining the sealed integrity of the motor compartment 40. Alternatively, the electronics components can be housed in a completely separate housing that is connected to the main body 24 through external wiring or cable assemblies. In such configurations, the separate electronics housing can be implanted adjacent to the growing rod 20 through the same surgical incision or through a nearby incision, with the connecting wiring routed subcutaneously between the devices. This modular approach can provide greater flexibility in device placement and may accommodate patients with specific anatomical constraints or surgical requirements that make integrated electronics placement challenging.

The dual antenna assembly 88 of the growing rod 20 provides a versatile configuration that enables the device to be implanted on either the left or right side of the patient's spine without compromising communication or power transfer capabilities. The circumferential windings 98 of the receive antenna 94 are wound around the ferrous core 92 in a manner that creates a substantially omnidirectional electromagnetic field pattern relative to the centerline C1 of the device. This omnidirectional characteristic allows the growing rod 20 to maintain effective electromagnetic coupling with the external controller 22 regardless of whether the device is positioned on the left or right side of the spinal column, eliminating the need for side-specific inventory, specialized left and right devices, or superior-inferior and inferior-superior “offset” device variants.

The antenna configuration also provides orientation flexibility that accommodates various surgical placement requirements and anatomical considerations. The growing rod 20 can be implanted in either a superior-to-inferior orientation or an inferior-to-superior orientation without affecting the electromagnetic coupling efficiency between the dual antenna assembly 88 and the external controller 22. This orientation independence is achieved through the symmetric electromagnetic field characteristics created by the circumferential winding pattern of the receive antenna 94, which maintains consistent coupling properties regardless of the device's rotational position about its longitudinal axis.

The electromagnetic coupling between the growing rod 20 and external controller 22 operates without polarity constraints, meaning that the power transfer and communication functions remain fully operational regardless of the relative positioning or orientation of the devices. This non-polarized operation simplifies the clinical procedure by allowing surgeons to position the growing rod 20 in the most anatomically appropriate configuration for each patient without concern for electromagnetic compatibility and without significant additional orientation constraints for the external controller. The robust coupling characteristics ensure that medical practitioners can achieve reliable wireless communication and power transfer across a wide range of device orientations and anatomical placements, enhancing the versatility and clinical utility of the system.

In an alternative embodiment, a dual-purpose module (e.g., 86) can be provided as a stand-alone device that is separate from the growing rod 20. In such an embodiment, the dual-purpose module can be independently implanted in the patient adjacent to the growing rod 20 and electrically coupled with the growing rod 20 (e.g., with a cable). The implantation of the dual-purpose module can occur through the same incision provided for the growing rod 20 or a nearby incision.

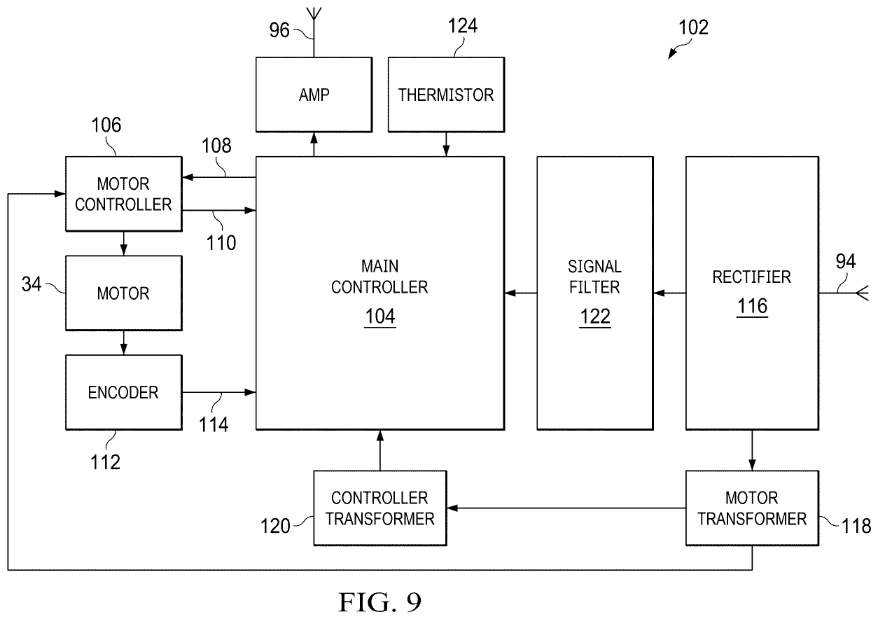

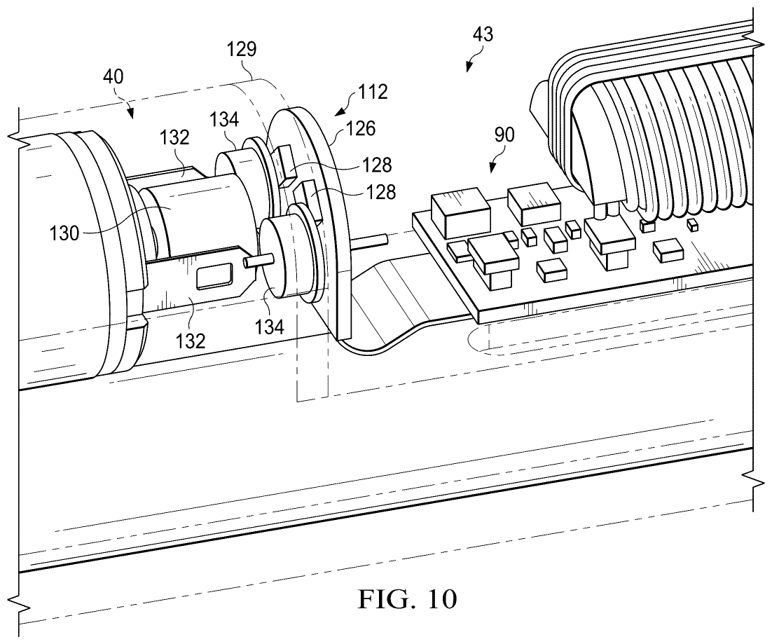

Referring now to FIGS. 8 and 9, an encoder 112 can be disposed at a distal end of the PCB 90 and can include a base plate 126 and a plurality of magnets 128 arranged radially about the base plate 126. The base plate 126 can be located adjacent to the motor 34, and a wall 129 can be disposed between the motor 34 and the base plate 126. The wall 129 can be disposed in the motor compartment 40 and can separate the motor compartment 40 from the electronics compartment 43. The motor 34 can include a rotary magnet 130 that is operably coupled with the output shaft 46 but disposed at an opposite end of the motor 34 as the output shaft 46. The rotary magnet 130 can rotate together with the output shaft 46. The magnets 128 of the encoder 112 can detect the position of the rotary magnet 130 to facilitate monitoring of the position and/or the angular velocity of the output shaft 46 which is provided as feedback data for use by the external controller 22, such as, for example, to control the speed of the motor 34 and/or to operate the motor 34 for specific number of revolutions to achieve a desired lengthening distance for the extension rod 28, as described above.

The encoder 112 can utilize multiple magnets 128 to provide comprehensive monitoring capabilities for the motor 34. In some embodiments, the plurality of magnets 128 can be positioned at predetermined angular intervals around the base plate 126, creating a magnetic pattern that can be detected as the rotary magnet 130 rotates with the output shaft 46. The multiple magnet configuration can provide redundancy that enhances the reliability of the encoder system. Further, magnetization or polarization patterns (e.g. encoded magnets) are envisioned and could be used to maintain magnetic coupling within specified distances between the coupling magnets. In the event that one of the magnets 128 experiences degradation or failure, the remaining magnets 128 can continue to provide position and speed feedback to the main controller 104. The encoder 112 may be configured to detect when a magnet 128 is not functioning properly by monitoring for missing or irregular signal patterns in the expected sequence of magnetic field variations.

When operating with a reduced number of functional magnets 128, the encoder 112 may automatically adjust its calculation algorithms to account for the larger angular intervals between active detection points. In some cases, the system can maintain adequate resolution for motor control purposes even with one or more failed magnets 128, though the precision of the position detection may be somewhat reduced or even impossible. This fault-tolerant design can help ensure continued operation of the growing rod 20 even in the presence of component degradation over the extended implantation period.

Referring now to FIG. 9, the motor compartment 40 can be configured as a “dry zone” that houses the motor 34 and its associated components. A dry zone may refer to a sealed environment that is substantially impervious to environmental contaminants such as bodily fluids, moisture, and other foreign substances that may be present in the implantation environment. The motor compartment 40 can be hermetically sealed to maintain this dry environment, which may be beneficial for protecting the motor 34 and its electrical components from corrosion, short circuits, and other forms of degradation that can occur when electrical components are exposed to conductive fluids or contaminants.

In contrast, the electronics compartment 43 can be configured as a “wet zone” that houses the PCB 90 and its associated components. A wet zone may refer to an environment that can potentially be exposed to environmental contaminants, including trace amounts of bodily fluids, humidity, or other substances that may infiltrate the compartment over time. The components housed within the electronics compartment 43, including the PCB 90, the dual antenna assembly 88, the encoder 112, and other associated electronics, can be wet-rated to withstand exposure to such environmental contaminants without experiencing premature failure or degradation.

The wall 129 can serve as a barrier between the motor compartment 40 and the electronics compartment 43, preventing contaminants that may enter the wet zone from reaching the motor 34 in the dry zone. This compartmentalization approach can allow for different levels of environmental protection to be applied to different components based on their specific requirements and vulnerability to contamination. In some instances, the components in the wet zone may be coated with protective materials, encapsulated in biocompatible compounds, covered with a rigid protective shell that physically shields the internal components from mechanical stress and environmental exposure, and/or manufactured using materials that are inherently resistant to the biological environment, while the motor 34 in the dry zone can rely primarily on the hermetic sealing for protection.

The motor 34 can include a pair of electrical posts 132 that can be electrically connected to terminals 134 that pass through the wall 129. The terminals 134 can be electrically coupled with the electronics package 102 to facilitate powering of the motor 34 from the power delivered to the receive antenna 94 from the external controller 22. The electrical posts 132 and terminals 134 effectively serve as an electrical feedthrough between the wet zone and the dry zone to facilitate electrical connection between the motor 34 and the dual-purpose module 86 for receiving power therefrom.

This feedthrough design minimizes the number of penetrations through the hermetic wall 129 while still allowing power to reach the motor 34 from the dual purpose module 86. By limiting the electrical connections to only those essential for operation, the design maintains the integrity of the hermetic seal between the wet and dry zones. In addition, the feedthrough is carefully designed to maintain the integrity of the hermetic seal of the dry zone that houses the motor 34 and its associated components. By limiting the number of penetrations through the wall 129 to only the essential electrical connections, the design reduces the risk of environmental contaminants migrating from the wet zone to the dry zone. The electrical posts 132 can be formed using biocompatible materials that can be effectively sealed to the wall 129, such as through welding techniques or other sealing methods that maintain the hermetic barrier while allowing electrical conductivity between the motor compartment 40 and the electronics compartment 40.

Although the encoder 112 is described herein as a magnetic-based encoder, alternative encoder designs are contemplated. In one example, a reflected light encoder can be implemented using a ceramic PCB positioned in the hermetic dry zone. This design includes an encoder disc with reflective engravings, with emitters sending light that reflects off the encoder disc and is detected by photo detectors mounted on the ceramic PCB with a brazed titanium ring. In another example, a transmitted light encoder can similarly utilize a ceramic PCB positioned in the hermetic dry zone, but operates by transmitting light through cutouts in an encoder disc positioned between the emitters and photo detectors. In yet another example, an optical motor encoder can also be implemented with emitter/receiver packages for position, speed, and direction detection. This design features an encoder disc that is painted and etched, attached to the motor output shaft, with four zones that alternate between absorbing and reflective properties. These optical encoder designs are particularly advantageous when operating under power budget constraints as they utilize lower power than magnetic-based encoders while still allowing position detection through the wall 129 that maintains the hermetic seal. The wall 129 can be configured with light pass through areas, such as transparent windows, optical lenses, fiber optic elements, or similar light-transmissive features that are hermetically sealed within the wall structure. These light pass through areas allow optical signals to traverse between the wet and dry zones while maintaining the integrity of the hermetic seal, enabling the optical encoders to function effectively without compromising the protection of sensitive electronic components in the dry zone.

Referring now to FIG. 10, the growing rod 20 can include an electronics package 102 that can power and control the operation of the growing rod 20 and communication with the external controller 22. It is to be appreciated that at least some of the electrical components of the electronics package 102 shown in FIG. 10 and described herein can be mounted on the PCB 90 and are represented in FIGS. 8 and 9 but are not identified by their corresponding reference numbers. The electronics package 102 can include a main controller 104 that is in communication with the motor 34 via a motor controller 106 that is coupled with the motor 34. The main controller 104 can control operation of the motor 34 via a control signal (e.g., a PWM signal) that is provided to the motor controller 106 via a signal line 108. A signal line 110 can report operational parameters of the motor 34, such as draw current or temperature, back to the main controller 104 for monitoring. If any of the operational parameters of the motor 34 exceed a predefined threshold, the main controller 104 can deactivate the motor 34 and can report the malfunction to the external controller 22 via the transmit antenna 96. The malfunction information can additionally or alternatively be encoded within the feedback signal that is transmitted from the growing rod 20 to the external controller 22, allowing the external controller 22 to receive diagnostic data and error conditions through the same communication channel used for operational status reporting. In some cases, the feedback signal may include specific error codes or diagnostic messages that identify the nature and severity of the detected malfunction, enabling the external controller 22 to provide appropriate alerts or corrective actions to the user.

The encoder 112 can be in communication with the main controller 104 via a signal line 114 for reporting the rotational position, angular velocity, direction of rotation, and operational status of the motor 34 (as feedback data) to the main controller 104. A rectifier 116 can be in communication with the receive antenna 94 and can be configured to extract the control data from the power signal generated by the external controller 22 and to deliver the power from the power signal to the motor 34 and the main controller 104 via transformers 118, 120, respectively. In some instances, the rectifier 116 can utilize sophisticated demodulation techniques to separate these dual functions. The control data from the rectifier 116 can be provided to the main controller 104 via a filter 122 that formats the control data for the main controller 104. The main controller 104 can be configured to control the operation of the growing rod 20 based upon the control data. For example, when the control data includes instructions for lengthening the extension rod 28 to a specific distance, the main controller 104 can operate the motor 34 in accordance with the instructions to achieve the specific distance. During operation of the motor 34, the main controller 104 can generate feedback data from the encoder 112. A thermistor 124 can be provided that is in communication with the main controller 104 for monitoring the internal temperature of the motor compartment 40. If the temperature exceeds a threshold value, the main controller 104 can transmit an error message to the external controller 22 via the transmit antenna 96 and can deactivate the electronics package 102.

The main controller 104 can serve as the central processing unit for the growing rod 20, orchestrating all operational functions and communication protocols within the implantable device. In some embodiments, the main controller 104 can be implemented as a microcontroller, microprocessor, digital signal processor (DSP), field-programmable gate array (FPGA), or application-specific integrated circuit (ASIC) that provides the computational capabilities necessary for device operation. The main controller 104 may manage motor control operations by generating control signals and monitoring operational parameters, process incoming communication data from the external controller 22 to execute lengthening commands, coordinate feedback data transmission to provide real-time status information, and implement safety protocols including force limiting and thermal monitoring. In some cases, the main controller 104 can perform signal processing functions to demodulate received power signals, encode feedback data for transmission, manage power distribution throughout the electronics package 102, and execute diagnostic routines to ensure proper device functionality. The main controller 104 can also handle device identification protocols, maintain operational logs, and coordinate with various sensors and components including the encoder 112, thermistor 124, and motor controller 106 to provide comprehensive control over the therapeutic functions of the growing rod 20 while maintaining safe operating conditions throughout the implantation period.

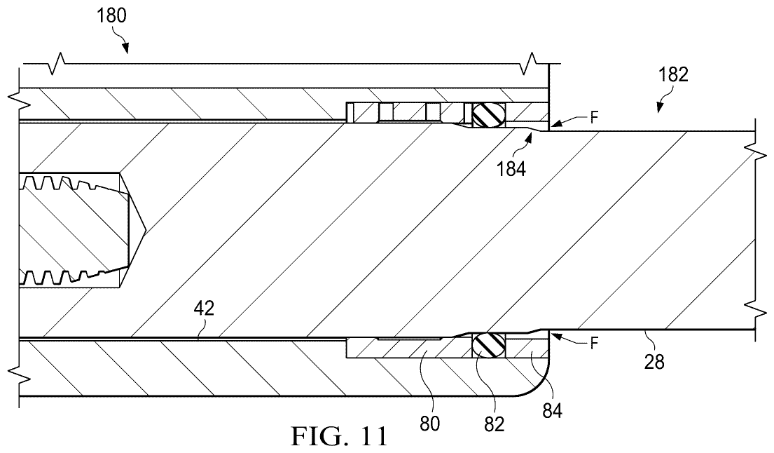

Referring now to FIG. 11, the growing rod 20 may incorporate additional functional capabilities beyond its primary lengthening mechanism to address the comprehensive requirements of implantable medical devices. For example, because the growing rod 20 is implantable into a patient's body, it must first be sterilized prior to implantation to eliminate any potential pathogens, microorganisms, or contaminants that could cause infection or adverse reactions in the patient. The sterilization process can be performed as one of the final steps of the manufacturing process which can include providing the growing rod 20 into a sealable pouch and imparting a sterilizing gas, such as ethylene oxide (ETO), Chlorine Dioxide, or hydrogen peroxide (H2O2) into the sealable pouch. The sterilizing gas is particularly effective for medical devices with complex geometries and sensitive electronic components that cannot withstand high-temperature steam sterilization methods. The gas molecules can penetrate small spaces and crevices within the device to ensure complete sterilization of all surfaces. The sterilization cycle typically involves a specific combination of gas concentration, humidity, temperature, and exposure time that has been validated to achieve a desired sterility assurance level. Once the growing rod 20 has been sterilized, the pouch can be sealed and delivered to a medical facility such that the growing rod 20 is ready for implantation when it is removed from the sealed pouch. As described above, the gear box compartment 41 and lower compartment 42 can potentially be exposed to the surrounding environment through the bushing 80 and the O-ring 82. As such, the extension rod 28 can be configured to be placed in a sterilization mode during sterilization that allows the second and third compartments 41, 42 to be sterilized by the sterilizing gas. This sterilization mode can be important for ensuring that all internal components that might come into contact with bodily fluids or tissues after implantation are properly sterilized, particularly those in the “wet zone” that are not hermetically sealed from the environment. The sterilization process can be carefully controlled to ensure that the electronic components in the motor compartment 40 are not damaged by the sterilizing agent, while still allowing adequate gas penetration into the gear box compartment 41 and lower compartment 42 where mechanical components are housed.

The extension rod 28 can be configured to be manipulated into a sterilization position that facilitates sterilization of the gear box compartment 41 and the lower compartment 42. This sterilization position may be a further retracted position beyond the retracted position shown in FIG. 2, allowing sterilizing agents to access internal components that would otherwise be sealed from the external environment. Once the sterilization process is complete, the extension rod 28 can be returned to the standard retracted position to restore the sealing function and prepare the device for clinical use.

As illustrated in FIG. 11, the extension rod 28 is shown to be provided in the sterilization position that allows for the sterilization of the gear box compartment 41 and lower compartment 42 with the sterilizing gas. The extension rod 28 is shown to include a proximal end 180, a distal end 182 and a tapered portion 184 that is disposed between the proximal end 180 and the distal end 182 and tapers inwardly towards the distal end 182. When the extension rod 28 is in either of the retracted or extended positions, as illustrated in FIGS. 2 and 3, the proximal end 180 can interface with the bushing 80 and the O-ring 82 to create an effective seal therebetween. When the extension rod 28 is in the sterilization position, as illustrated in FIG. 11, the tapered portion 184 can be withdrawn from the O-ring 82 to create a flow path F between the extension rod 28 and the O-ring 82 that allows the sterilization fluid that is imparted to the growing rod 20 to flow past the bushing 80 and the O-ring 82 and into the gear box compartment 41 and the lower compartment 42 to facilitate sterilization thereof. Once the sterilization of the growing rod 20 is complete, the extension rod 28 can be returned to the retracted position shown in FIG. 2, and the growing rod 20 can be sealed within the sealable pouch that is delivered to the medical facility.

The sterilization process involves placing the growing rod 20 with the extension rod 28 in the sterilization position into a sealable pouch. In this configuration, the tapered portion 184 is withdrawn from the O-ring 82, creating a flow path F that allows sterilizing gas to penetrate the gear box compartment 41 and lower compartment 42. The growing rod 20 can be assembled directly in the sterilization position during manufacturing, or alternatively, may be moved to the sterilization position using a specialized external controller that includes restricted software functionality not available to commercial users. This specialized external controller can wirelessly power and command the growing rod 20 to retract the extension rod 28 into the sterilization position, enabling proper gas flow for sterilization of the wet zone components. The sterilization parameters, including gas concentration, humidity levels, temperature, and exposure duration, are carefully controlled to achieve the required sterility assurance level while protecting the electronic components in the hermetically sealed motor compartment 40.

During the sterilization cycle, the sterilizing agent (e.g., gas) can be introduced into the sealed pouch containing the growing rod 20 at predetermined concentrations and pressures. The sterilizing agent can be introduced gradually to allow for uniform distribution throughout the pouch and penetration into the gear box compartment 41 and lower compartments 42 through the flow path F created by the tapered portion 184. The sterilizing agent can remain in contact with all surfaces for a specified dwell time to ensure effective sterilization of all components within the wet zone. Following the sterilization exposure period, the sterilizing agent can be evacuated from the pouch through a controlled venting process that may include multiple evacuation and purge cycles to remove residual sterilizing agent. The evacuation process can be followed by an aeration period where the growing rod 20 remains in the sealed environment to allow any remaining gas residues to dissipate to acceptable levels before the device is considered ready for distribution. This aeration phase may be useful to ensure that no harmful residues remain that could affect patient safety upon implantation.

Following sterilization and aeration to remove residual gas, the specialized version of the external controller 22 can wirelessly power and command the growing rod 20 through the sterile packaging to move the extension rod 28 from the sterilization position to a starting position. This starting position can effectively be the retracted position where the extension rod 28 is sealably engaged with the O-ring 82 and the bushing 80 to restore the sealing function of the growing rod 20 such that the growing rod 20 is a “finished good” that is ready for implantation.

Once in this “finished good” position, the growing rod 20 is ready for distribution, and the extension rod 28 can only be lengthened from this position, not retracted back to the sterilization position by a user. This functionality is maintained through recording of motor revolutions, historical total distraction, and software control parameters. This sterilization process may not be achievable without the wireless connectivity and powering capabilities of the growing rod 20, as the ability to remotely command the extension rod 28 through the sealed sterile packaging eliminates the need to breach the sterile barrier for mechanical adjustment. This process also ensures that all internal components that might come into contact with bodily fluids after implantation are properly sterilized, particularly those in the “wet zone” that are not hermetically sealed from the environment, while maintaining the integrity of the sensitive electronic components.

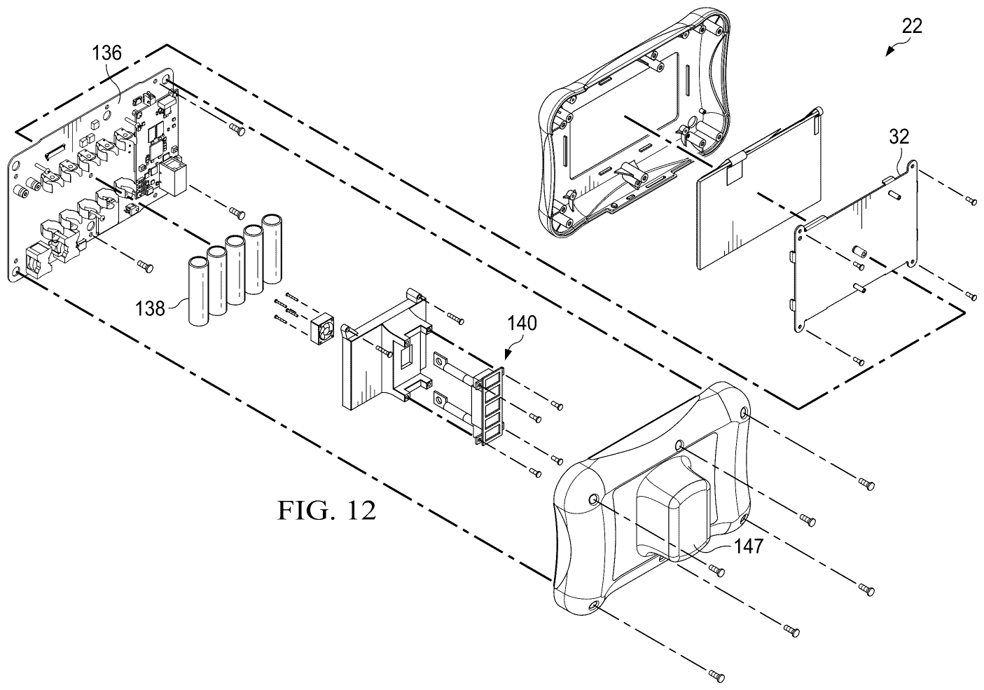

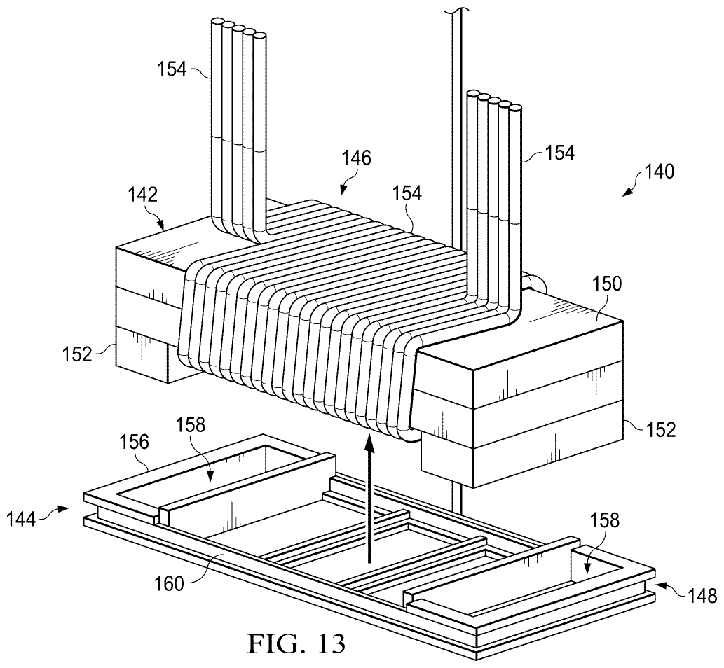

Having described the growing rod 20 and its various operational capabilities, attention is now directed to the external controller 22 and its components that enable wireless communication and power delivery to the implanted device. The external controller 22 can incorporate specialized hardware that facilitate communication with and control over the growing rod 20 while generating a user-friendly interface for medical professionals. Referring now to FIG. 12, the external controller 22 is shown to include a (printed circuit board) PCB 136, a battery pack 138, and a dual antenna assembly 140. The battery pack 138 can be mounted to the PCB 136 and can be configured to store electrical energy for powering the external controller 22. The dual antenna assembly 140 can also be mounted to the PCB 136 and can be configured to facilitate bidirectional communication with the growing rod 20. The display 32 can be mounted to the PCB 136 on an opposite side as the battery pack 138 and the dual antenna assembly 140.

The display 32 can be implemented as a touchscreen display that provides both visual output and tactile input capabilities. In some embodiments, the touchscreen display may utilize capacitive touch sensing technology to detect user interactions with the display surface. The touchscreen functionality may allow users to directly interact with displayed interface elements by touching, tapping, or gesturing on the screen surface. The display 32 can present various graphical user interface elements such as buttons, menus, input fields, status indicators, and data visualization components. Users may navigate through different screens or interface modes by touching appropriate areas of the display. In some cases, the touchscreen may support multi-touch gestures such as pinching, swiping, or scrolling to facilitate intuitive user interaction with the displayed content.

The display 32 can be configured to receive user inputs through touch interactions, which can include selection of operational parameters, confirmation of commands, navigation between different functional modes, and entry of alphanumeric data. The touchscreen interface may provide immediate visual feedback to user interactions, such as highlighting selected items or displaying confirmation messages. The touchscreen display may utilize various sensing technologies including capacitive touch sensing, resistive touch sensing, infrared touch detection, or surface acoustic wave technology to detect and respond to user interactions with the display surface. In some instances, the touchscreen display 32 may incorporate haptic feedback mechanisms that provide tactile responses to user touches, enhancing the user experience by confirming successful input registration. The touchscreen display 32 may be utilized for any of the various input and output functions described throughout the embodiments disclosed herein, providing a versatile interface for controlling and monitoring the operation of the growing rod 20 and external controller 22 system.

In alternative embodiments, the display 32 may be implemented as a non-touchscreen display with separate input mechanisms. These may include tactilely distinguishable pushbuttons positioned around the display that provide feedback when pressed, or an integrated keyboard for more complex data entry. The pushbuttons may be arranged as directional navigation controls, selection buttons, or function-specific buttons, potentially with backlighting or visual indicators to show their current state. The keyboard option may include alphanumeric keys, function keys, and dedicated operation buttons that allow users to enter specific commands, numerical values, or device identifiers directly. In such embodiments, the display 32 would present menu systems, operational parameters, and status information that users navigate using these input mechanisms. Visual cues such as highlighting, cursor positioning, or selection indicators would correspond to the current focus as controlled by user inputs. These alternative input methods may support different interaction styles, from sequential button navigation to direct command entry, providing flexibility in how users control the growing rod 20 through the external controller 22.

Referring now to FIG. 13, the dual antenna assembly 140 can include a ferrous core 142, a bobbin 144, a transmit antenna 146 for transmitting the modulated power signal to the growing rod 20, and a receive antenna 148 for receiving the feedback signal from the growing rod 20. The receive antenna 148 and the transmit antenna 146 can be configured to receive and transmit signals simultaneously when interacting with the growing rod 20.

The ferrous core 142 can include a central portion 150 and a pair of leg portions 152 that extend downwardly from the central portion 150. The transmit antenna 146 can include windings 154 that are circumferentially wound around the central portion 150. The bobbin 144 can be made of similar or the same ferrous material as the ferrous core 142 and can include an outer perimeter 156 and a pair of receptacles 158. The receive antenna 148 can include windings 160 that are wound circumferentially around the outer perimeter 156 of the bobbin 144. The leg portions 152 of the ferrous core 142 can extend into the receptacles 158 of the bobbin 144 and can be attached thereto such that the ferrous core 142 and the bobbin 144 form a unitary core.

The windings 154 of the transmit antenna 146 and the windings 160 of the receive antenna 148 can be substantially orthogonal to each other which can alleviate cross-talk, interference, malalignment sensitivity (e.g., translation and rotation), and other drawbacks that are oftentimes associated with simultaneously operating antennas that are in close proximity with one another. The windings 154, 160 can be formed of a coated magnetic wire such as, for example, thermoplastic coated copper wire. Because the transmit antenna 146 is responsible for transmitting a power signal, the gauge of the wire used for the windings 154 of the transmit antenna 146 can be greater than the gauge of the wire used for the windings 160 of the receive antenna 148 which is only responsible for receiving a data signal (e.g., the feedback signal) from the growing rod 20. Using thinner wire for the windings 160 can allow for a greater quantity of turns to be incorporated into the allotted space for the transmit antenna 96 which can enhance the reception of the feedback signal and can strengthen the electromagnetic field which can result in improved communication with the growing rod 20.

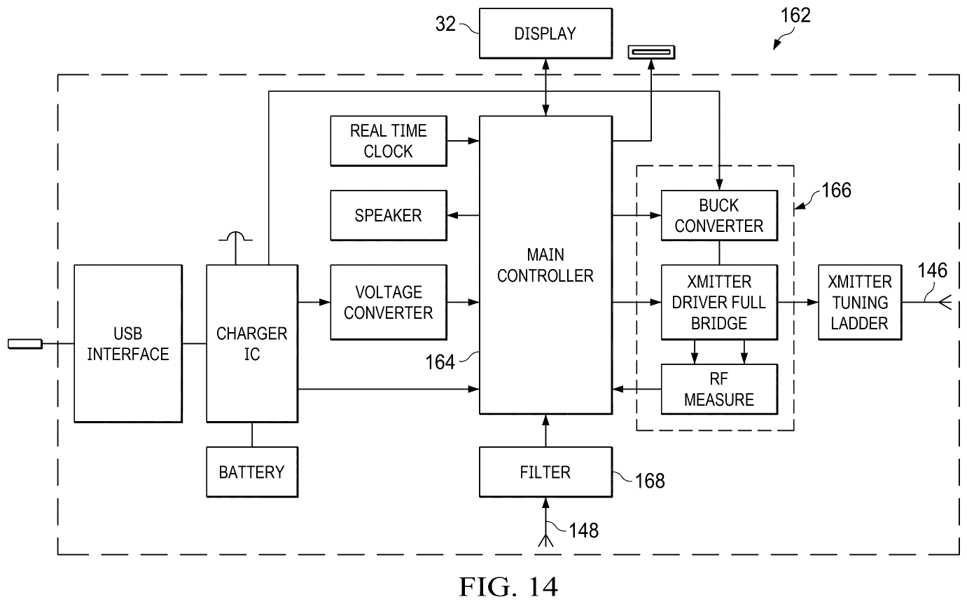

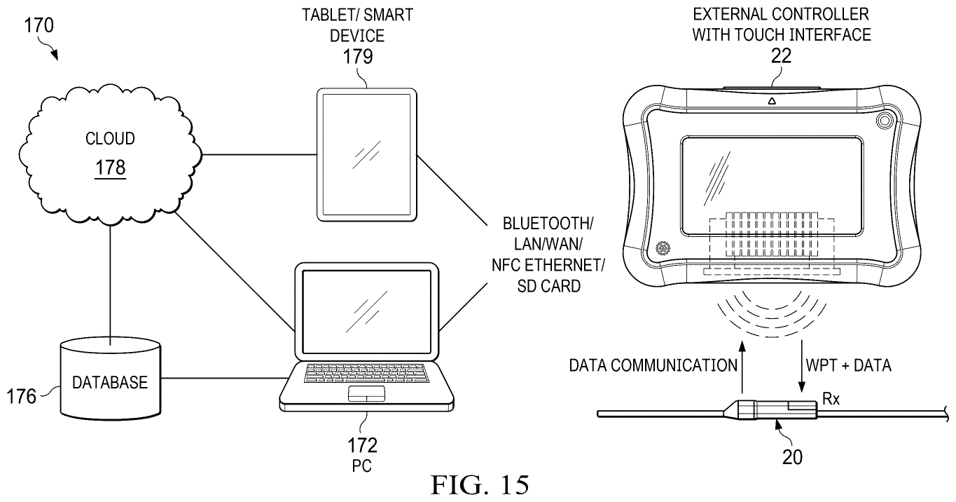

Referring now to FIG. 14, the external controller 22 can include an electronics package 162 that controls the operation of the external controller 22 and the communication with the growing rod 20. It is to be appreciated that at least some of the electrical components of the electronics package 162 shown in FIG. 14 and described herein can be mounted on the PCB 136 and are represented in FIG. 12 but are not identified by their corresponding reference numbers. The electronics package 162 can include a main controller 164 that is in communication with the display 32 and configured to communicate with the display 32 to receive information that is entered on the display 32 from a user as well as to generate information for presentation on the display 32. A communication package 166 can be in communication with the main controller 164 and can be configured to facilitate generation of the modulated power signal and modulation of the control data onto the power signal for delivery to the growing rod 20. The communication package 166 can include specialized circuitry for generating carrier signals at precise frequencies, modulation components for embedding digital control data onto the power signal, and power amplification stages that ensure sufficient energy transmission to the implanted growing rod 20. In some embodiments, the communication package 166 may incorporate adaptive frequency tuning capabilities that allow it to automatically adjust the carrier frequency to match the resonant characteristics of the growing rod's receive antenna 94, thereby optimizing power transfer efficiency across varying implantation depths and tissue compositions.

As will be described in further detail below, the main controller 164 can tailor the characteristics of the power signal to the growing rod 20. The feedback data from the growing rod 20 that is received at the receive antenna 148 can be provided to the main controller 164 via a filter 168. The filter 168 can be designed to isolate the feedback signal from electromagnetic interference and noise that may be present in the operating environment, ensuring reliable data reception from the implanted growing rod 20. The filter 168 may incorporate multiple filtering stages including bandpass filtering to isolate the specific frequency band used for feedback transmission, signal conditioning circuitry to normalize signal levels, and digital processing elements that perform error detection and correction on the received data stream. In some embodiments, the filter 168 may also include adaptive filtering capabilities that can automatically adjust to changing signal conditions, compensating for variations in coupling efficiency or interference levels that might occur during a lengthening procedure as the patient or external controller 22 position shifts slightly. This sophisticated filtering approach helps maintain consistent and reliable communication with the growing rod 20 even in challenging clinical environments where various electronic devices may be operating simultaneously.

The external controller 22 can incorporate onboard data storage capabilities that enable comprehensive data collection and logging throughout treatment sessions. The onboard data storage capabilities may utilize solid-state drives (SSDs), embedded flash memory, secure digital (SD) card slots, or non-volatile random-access memory (NVRAM). The external controller 22 may combine these technologies, such as using internal flash memory for system operation while employing removable SD cards for treatment logs and patient data that can be transferred to other systems for analysis.

The external controller 22 can collect and store various types of operational data including treatment session logs that document the date, time, duration, and parameters of each lengthening procedure, device performance metrics such as power consumption, signal strength measurements, and communication quality indicators, patient-specific information including device identifiers and treatment protocols, diagnostic data from the growing rod 20 such as motor performance, temperature readings, and force measurements, and error logs that capture any fault conditions or operational anomalies encountered during treatment sessions. This comprehensive data collection capability may enable medical professionals to track treatment progress over time, analyze device performance trends, and maintain detailed records for clinical documentation and regulatory compliance purposes.

The external controller 22 can be configured with networking capabilities that allow it to communicate with hospital information systems, electronic medical records, or remote monitoring platforms through various connectivity options. In some embodiments, the external controller 22 can include wireless networking capabilities such as Wi-Fi, Bluetooth, or cellular connectivity that enable real-time data transmission to remote databases or cloud-based storage systems. The networking functionality can allow for automatic synchronization of treatment data with hospital systems, remote monitoring by medical professionals, software updates and firmware upgrades, and integration with broader healthcare information technology infrastructure. The networked configuration can also enable multi-site data sharing for research purposes, remote technical support, and centralized device management across multiple clinical locations.

In healthcare environments where networking may be restricted due to privacy concerns, regulatory requirements, or security policies, the external controller 22 may alternatively utilize removable storage solutions such as SD cards, USB drives, or other portable memory devices. The removable storage approach may allow medical professionals to physically transfer collected data from the external controller 22 to authorized computer systems for analysis, archival, or integration with patient records while maintaining strict control over data access and transmission. The removable storage may be encrypted to protect patient privacy and may include authentication mechanisms to ensure data integrity during transfer. This approach may provide healthcare facilities with flexibility to maintain compliance with privacy regulations while still enabling comprehensive data collection and analysis capabilities for treatment optimization and clinical research purposes.

Having described the hardware components of both the growing rod 20 and external controller 22, the sophisticated communication protocol that enables their interaction will now be described. The external controller 22 can deliver power to the growing rod 20 via the power signal through electromagnetic induction. The electromagnetic induction can facilitate electrical coupling between the external controller 22 and the growing rod 20 when the devices are positioned within sufficient proximity to enable adequate power transfer levels. When the external controller 22 is brought close enough to the implanted growing rod 20, the alternating magnetic field generated by the transmit antenna 146 can induce sufficient electrical currents in the receive antenna 94 to power the motor 34, electronics package 102, and other onboard components of the growing rod 20 from the transmitted power signal. This inductive coupling approach can allow for efficient power transfer without requiring direct physical contact between the devices, while the strength and efficiency of the coupling can be optimized through proper alignment and frequency tuning between the transmit and receive antennas. The physical contact between devices may involve either a wireless or corded arrangement for charging the external controller. A wireless controller is envisioned to provide greater ease of use and device positioning. Position of the external controller 22 to the spine involves specific designs to minimize the distance. One such design includes a protrusion 147 for the antenna allowing greater fit into significant lordosis curvature of the spine, and reduce the user's hands from creating an obstruction or increasing an air gap for electromagnetic coupling. The design of the external controller 22 can further prevent inadvertent connection with adjacent rods all while enhancing the proximity between the external controller 22 and growing rod 20.

The power signal can be modulated (e.g., by the communication package 166) to carry the communication data such that both power and data are transmitted simultaneously by the power signal to the growing rod 20. The modulation process can embed the digital control data onto the carrier signal using various encoding techniques that maintain power delivery efficiency while enabling reliable data transmission through biological tissue. The power signal can operate at a carrier frequency that is tuned to match the resonant characteristics of the receive antenna 94 of the growing rod 20. In one embodiment, the carrier frequency can be between about 100 kHz and 500 kHz and in some instances between about 290 kHz and 390 kHz enhance the penetration through biological tissue while maintaining efficient power transfer across varying implantation depths and tissue compositions.

The power signal can be modulated using any of a variety of modulation techniques that can be selected based on specific communication requirements, environmental conditions, and operational parameters. In one instance, the power signal can be modulated using amplitude shift keying (ASK), where the amplitude of the carrier signal is varied to represent different binary data states. The communication package 166 may implement ASK by switching between distinct amplitude levels—a higher amplitude representing one binary state and a lower amplitude representing the other. The modulation depth can be adjusted to optimize the balance between data transmission reliability and power delivery efficiency, with deeper modulation providing enhanced data discrimination at the growing rod 20.

Alternative modulation techniques may include frequency shift keying (FSK), where the carrier frequency shifts between discrete values to represent different data states, providing enhanced noise immunity in environments where signal amplitude may be affected by coupling variations. On-off shift keying (OOK) can be utilized for simple and robust data transmission, where the presence or absence of the carrier signal represents different data states. When employing this modulation technique, the duty cycle and timing parameters can be optimized to maintain sufficient average power delivery while enabling clear data detection. Phase shift keying (PSK) may be employed to vary the phase of the carrier signal for encoding data, offering improved spectral efficiency and reduced susceptibility to amplitude variations. When employing this modulation technique, the phase shifts can be selected to provide reliable data detection while minimizing impact on power transfer efficiency. In other instances, quadrature amplitude modulation (QAM) can be utilized and can combine both amplitude and phase modulation to encode multiple bits per symbol, enabling higher data transmission rates while maintaining the same carrier frequency.

When the power signal is received by the growing rod 20, the electronics package 102 can be responsible for extracting both power and data from the same received signal through sophisticated demodulation techniques. The modulated power signal is transmitted through the transmit antenna 146 of the external controller 22 and received by the receive antenna 94 of the growing rod 20. The growing rod 20 demodulates the received power signal to extract both electrical energy and embedded control data. In particular, the rectifier 116 within the electronics package 102 separates these dual functions using sophisticated demodulation techniques. The extracted electrical energy is distributed to power the motor 34 and other components through the motor transformer 118 and controller transformer 120. Simultaneously, the extracted control data is processed through the signal filter 122 that implements multiple filtering stages including bandpass filtering to isolate specific frequency components, low-pass filtering to remove high-frequency noise, and digital signal processing algorithms to enhance signal quality and reduce interference before delivery to the main controller 104. This dual-purpose approach for separating the power from the communication data can eliminate the need for separate power and data transmission channels, simplifying the wireless interface while ensuring reliable operation of the implanted device.

The modulation components of the external controller 22 and the demodulation components of the growing rod 20 can be configured with matching protocols to ensure reliable data transmission and reception. The communication package 166 of the external controller 22 can implement specific modulation parameters that correspond directly to the demodulation capabilities of the rectifier 116 and signal filter 122 within the growing rod 20. In some instances, the external controller 22 may store multiple modulation protocol configurations in its main controller 164, allowing it to select the appropriate protocol based on the specific growing rod model, implantation conditions, or communication requirements. For example, for challenging implantation environments where signal attenuation is high, the system may employ more robust modulation schemes such as FSK with wider frequency separation or PSK with enhanced error correction coding. In instances where power transfer efficiency is prioritized over data transmission speed, the protocol may utilize lower modulation depths or longer symbol periods to maintain adequate power delivery while ensuring reliable data reception.

The system 23 may also implement adaptive protocol selection, where the external controller 22 can automatically adjust the modulation parameters based on real-time feedback from the growing rod 20 regarding signal quality, power reception efficiency, and communication reliability. In some instances, the protocol matching may include synchronization sequences that allow the devices to establish and maintain proper timing alignment throughout the communication session. The external controller 22 may transmit specific preamble patterns that enable the growing rod 20 to lock onto the carrier frequency and establish proper demodulation timing. Additionally, the protocols may incorporate automatic gain control coordination, where the external controller 22 adjusts its transmission power based on feedback regarding the received signal strength at the growing rod 20, ensuring optimal signal levels for reliable demodulation while minimizing power consumption.

The feedback signal transmission operates independently of the power signal delivery through a separate communication channel than the communication channel that is responsible for receiving the power signal. The main controller 104 of the growing rod 20 generates the feedback data and transmits it via a feedback signal through the transmit antenna 96 at a feedback frequency that differs from the power signal carrier frequency. In one example, the feedback frequency can be about 3.7 MHz, which can effectively reduce interference between power delivery and data feedback channels. This frequency separation allows for simultaneous bidirectional communication without compromising either power transfer efficiency or data transmission reliability.

The feedback signal may employ on-off shift keying (OOK) protocol for optimal power efficiency and reliable data transmission through biological tissue. In this implementation, the feedback data is encoded by switching the transmission on and off according to the binary data pattern. The electronics package 102 can incorporate a voltage controlled oscillator (VCO) with standard UART input to reduce signal noise that might otherwise be present from the simultaneously received power signal. This modulation technique requires minimal power for signal generation while providing adequate signal-to-noise ratio for reliable communication through tissue.

The feedback signal can be received by the receive antenna 148 of the external controller 22 and processed through the filter 168 before delivery to the main controller 164. The filter 168 can be designed to isolate the feedback signal from electromagnetic interference and noise that may be present in the operating environment, ensuring reliable data reception from the implanted growing rod 20. The filter 168 can incorporate multiple filtering stages including bandpass filtering to isolate the specific frequency band used for feedback transmission, signal conditioning circuitry to normalize signal levels, and digital processing elements that perform error detection and correction on the received data stream.

The external controller 22 and growing rod 20 may implement various optimization techniques to enhance the reliability and efficiency of wireless power transfer and communication throughout the treatment process. These optimization approaches can adapt to changing conditions and environmental factors that may affect the electromagnetic coupling between the devices during clinical use.

One such optimization technique is a frequency optimization algorithm that the external controller 22 can employ to help optimize the power transfer efficiency between the external controller 22 and the growing rod 20. The frequency optimization algorithm can be implemented to automatically determine the optimal carrier frequency for the power signal by systematically evaluating the coupling efficiency across a predetermined frequency range. This algorithm can be particularly valuable in clinical applications where variations in tissue composition, implantation depth, and device positioning may affect the resonant characteristics of the wireless power transfer system.

The execution of the frequency optimization algorithm can begin by the external controller 22 first establishing a baseline frequency range that encompasses the expected resonant frequency of the receive antenna 94 within the growing rod 20. The expected resonant frequency can be determined by interrogating the growing rod 20, from previous calibration procedures, or from understood manufacturing specifications that correlate antenna characteristics with resonant frequency parameters for the particular implanted device. In some instances, this frequency range can be between about 100 kHz and 500 kHz and in some instances between about 290 kHz and 390 kHz, though the specific range can be adjusted based on the particular antenna design and expected operating conditions.

Once the baseline frequency range has been established, the external controller 22 can divide this frequency range into discrete frequency steps, with the step size selected to provide adequate resolution for identifying the optimal frequency while maintaining reasonable search time duration. The external controller 22 can then conduct a frequency sweep by transmitting test signals at each frequency step within the predetermined range while monitoring the feedback data received from the growing rod 20. The feedback data can include power reception efficiency metrics, signal quality indicators, and coupling strength measurements that enable the external controller 22 to evaluate the effectiveness of power transfer at each tested frequency. The growing rod 20 can measure various parameters including received signal strength, power conversion efficiency, and signal-to-noise ratio, transmitting this information back to the external controller 22 through the feedback signal channel.

During the frequency sweep, the external controller 22 can implement a systematic approach where it sequentially transmits at each frequency step for a predetermined duration, allowing sufficient time for the growing rod 20 to stabilize its measurements and transmit accurate feedback data. During each frequency step, the external controller 22 can record the corresponding feedback metrics and associate them with the specific test frequency. This process continues until the entire frequency range has been evaluated/searched, creating a comprehensive profile of coupling efficiency versus frequency.

In some instances, executing the frequency sweep can include employing adaptive step sizing to optimize the search process. In these instances, the external controller 22 can begin with relatively large frequency steps to quickly identify regions of improved coupling efficiency, then reduce the step size in promising frequency ranges to achieve finer resolution. This adaptive approach can reduce the overall search time while maintaining the precision necessary to identify the optimal operating frequency.

Once the sweep is complete, the external controller 22 can then analyze the collected frequency response data to identify the frequency that provides the highest power transfer efficiency. This analysis can involve comparing the feedback metrics across all tested frequencies and selecting the frequency that yields the best combination of power reception efficiency, signal quality, and coupling stability. In some cases, the algorithm may apply weighting factors to different metrics based on their relative importance for the specific application or operating conditions.

The frequency optimization algorithm can also incorporate hysteresis or stability criteria to prevent frequent frequency changes due to minor variations in coupling conditions. The algorithm can require that a new frequency demonstrate a significant improvement over the current operating frequency before initiating a frequency change. This approach can help maintain stable operation while still allowing the system to adapt to substantial changes in coupling conditions.

In some embodiments, the frequency optimization algorithm can be executed automatically at predetermined intervals during operation to compensate for gradual changes in coupling conditions that may occur due to patient movement, tissue changes, or device positioning variations. The algorithm can also be triggered manually by the user or automatically when the system detects degraded coupling efficiency or communication quality.

The frequency optimization process may include safety protocols to ensure continuous power delivery to the growing rod 20 during the execution of the algorithm. In particular, the algorithm can maintain minimum power levels at all tested frequencies to prevent interruption of device operation, while still providing sufficient signal variation to enable accurate efficiency measurements. In some cases, the search algorithm can be designed to complete within a specified time limit to minimize any potential disruption to ongoing therapeutic procedures.

Another optimization technique that can be employed is a power management routine that the growing rod 20 and the external controller 22 can cooperatively utilize to optimize power consumption and extend operational efficiency throughout treatment sessions. The power management routine can facilitate dynamic adjustment of power delivery parameters based on real-time operational requirements and device feedback to maintain optimal performance while conserving energy resources.

The power management routine can begin with the external controller 22 establishing baseline power requirements for the growing rod 20 based on the specific operational mode and treatment parameters. The external controller 22 can determine initial power levels by analyzing feedback data from the growing rod 20 that includes current motor load requirements, internal resistance measurements, and operational status indicators. This baseline assessment allows the system to establish appropriate starting power levels that provide adequate energy for device operation while avoiding unnecessary power waste. In one embodiment, the external controller 22 can implement a buck converter circuit that efficiently steps down voltage from the battery pack 138 to the appropriate level required for power transmission, with the initial duty cycle set based on these baseline requirements.

During operation, the growing rod 20 can continuously monitor its internal power consumption through various sensors and measurement circuits within the electronics package 102. The main controller 104 can track motor current draw, voltage levels across different components, and overall power efficiency metrics. This monitoring data can be transmitted to the external controller 22 through the feedback signal, providing real-time information about the power requirements and consumption patterns of the implanted device. For buck converter topologies, the feedback data includes specific voltage measurements that enable the external controller 22 to precisely adjust the buck converter's duty cycle to maintain optimal power delivery.

The external controller 22 can process the received power consumption feedback data and implement adaptive power adjustment algorithms that modify transmission parameters to optimize energy delivery. When the feedback data indicates that the growing rod 20 requires higher power levels, such as during periods of increased mechanical resistance or motor load, the external controller 22 can automatically increase the amplitude of the power signal or adjust the duty cycle of the power transmission (e.g., via the buck converter), effectively increasing the amplitude of the power signal to provide additional energy. Conversely, when the feedback data shows lower power requirements, such as during idle periods or reduced mechanical loading, the external controller 22 can decrease power transmission levels (e.g., via the buck converter) to conserve energy and reduce unnecessary electromagnetic field exposure.

The power management routine can incorporate predictive algorithms that anticipate power requirements based on operational patterns and treatment protocols. The external controller 22 can analyze historical power consumption data and treatment session patterns to predict upcoming power needs and preemptively adjust transmission parameters (e.g., the buck converter's duty cycle). This predictive approach can help maintain consistent power delivery while minimizing energy waste during transitions between different operational phases. The system implements dynamic buck modulation, where the duty cycle is continuously adjusted in real-time rather than using fixed preset levels, allowing for precise power control that responds to changing conditions within milliseconds.

In some instances, the power management routine can implement load-based power scaling where the external controller 22 adjusts power delivery (e.g., the buck converter's duty cycle) based on the mechanical load experienced by the motor 34 within the growing rod 20. When the feedback data indicates increased motor current draw due to higher spinal resistance or mechanical binding, the system can automatically increase power transmission duty cycle (e.g., at the buck converter) to maintain adequate torque and extension force. The power scaling algorithm can correlate motor current measurements with required power level duty cycle adjustments, enabling precise power delivery that matches the instantaneous mechanical demands of the lengthening procedure while maintaining optimal efficiency of the buck converter circuit.

The power management routine can also include thermal management protocols that adjust power delivery (e.g., the buck converter's duty cycle) based on temperature feedback from the thermistor 124 within the growing rod 20. When the feedback data indicates elevated internal temperatures, the external controller 22 can implement power reduction strategies such as decreasing transmission amplitude, implementing duty cycle modulation, or initiating cooling periods between operational cycles, implementing pulse-width modulation with variable on/off periods, or initiating cooling periods between operational cycles where the duty cycle is reduced to minimal levels. These thermal management protocols can help prevent overheating while maintaining safe operating conditions throughout extended treatment sessions.

The external controller 22 can also implement battery conservation algorithms that optimize its own power consumption based on the remaining battery capacity in the battery pack 138. When battery levels decrease below predetermined thresholds, the system can automatically adjust operational parameters to extend treatment session duration while maintaining sufficient power for essential operations. The dynamic buck modulation system can implement high-efficiency modes that optimize the duty cycle specifically for low battery conditions, which balances power delivery with minimal battery drain. These adjustments may also include reducing display brightness on the display 32, optimizing communication protocols to minimize power consumption, implementing power-saving modes during idle periods, and adjusting the frequency and duration of diagnostic communications with the growing rod 20.

In some cases, the power management routine can incorporate adaptive coupling optimization that adjusts power transmission parameters (e.g., the buck converter's duty cycle) based on the electromagnetic coupling efficiency between the external controller 22 and the growing rod 20. When the feedback data indicates poor coupling efficiency due to device positioning or tissue characteristics, the external controller 22 can increase transmission power to compensate for coupling losses while maintaining adequate power delivery to the implanted device. For buck converter topologies, the dynamic buck modulation system continuously adjusts the duty cycle in real-time based on coupling efficiency measurements, with adjustments occurring as frequently as 100 times per second to maintain optimal power transfer despite minor movements or positioning changes. The system can also provide guidance to the user through the display 32 to optimize device positioning and improve coupling efficiency, thereby reducing overall power requirements.

The power management routine can also include emergency power procedures that ensure continued operation during critical treatment phases even when power resources are limited. The external controller 22 can identify when a low power condition exists and in response can prioritize power delivery to essential functions such as motor operation and safety monitoring while temporarily reducing power to non-critical systems such as advanced diagnostic features or communication enhancements. In emergency conditions with buck converter topologies, the dynamic buck modulation system can implement an ultra-efficient duty cycle profile that maintains a minimum viable power level, typically operating at a carefully optimized 50-55% duty cycle that maximizes the energy transfer efficiency of the buck converter circuit while minimizing battery consumption. The emergency power procedure can help ensure that lengthening procedures can be completed safely even when the battery pack 138 is depleted or coupling conditions between the growing rod 20 and the external controller 22 are suboptimal.

Other optimization techniques that might be employed include automatic gain control (AGC), error detection and correction algorithms, signal synchronization, adaptive capabilities, and time-division multiplexing. The AGC technique can maintain consistent signal levels despite variations in received power strength, automatically adjusting amplification levels to optimize signal-to-noise ratio while preventing signal saturation. The error detection and correction technique, including cyclic redundancy check (CRC) techniques and forward error correction (FEC) coding, can be employed to ensure reliable data recovery from transmitted signals despite electromagnetic interference or coupling variations. The signal synchronization techniques can be employed to maintain proper timing alignment between transmitted and received signals through clock recovery circuits that extract timing information from received signals. This technique can enhance accurate data sampling and demodulation, particularly during extended procedures where maintaining reliable communication is critical for safe operation.

The adaptive capabilities can be employed for the communication protocol to automatically adjust signal parameters based on real-time assessment of communication quality and coupling conditions. In particular, the growing rod 20 can continuously monitor various signal quality metrics through the main controller 104 and can transmit this information as part of the feedback data, including signal strength measurements, signal-to-noise ratio values, power reception efficiency metrics, and coupling quality indicators. The external controller 22 can process this feedback data through its main controller 164 and can automatically adjust transmission power levels, modify carrier frequencies, alter modulation parameters, or provide real-time guidance to the user through the display 32 for optimal device positioning and alignment.

The time-division multiplexing techniques can be utilized to transmit different types of feedback data sequentially, allowing efficient use of available communication bandwidth while maintaining data integrity. The system can implement error detection and correction protocols specifically for critical feedback information to ensure accurate reception by the external controller 22, particularly in challenging transmission environments where signal degradation may occur due to tissue characteristics or device positioning variations.

Beyond the power management and frequency optimization capabilities described above, the system 23 can implement a distributed control architecture where certain control functions are redundantly implemented on both the external controller 22 and the growing rod 20 to ensure optimal performance despite communication latency inherent in wireless power and data transmission. This redundant approach enables the growing rod 20 to autonomously execute control functions that are normally managed by the external controller 22 during periods when real-time communication may be insufficient for immediate response requirements.

The distributed control architecture operates with overlapping control capabilities between the external controller 22 and the growing rod 20. The external controller 22 can manage primary control functions including treatment protocol execution, power delivery optimization, and overall system coordination through the communication data transmitted via the modulated power signal. However, the growing rod 20 can maintain a subset of the control functions also provided by the external controller 22. This subset of control functions are therefore redundant implementations of the primary control functions at the external controller 22 and are typically time-critical control functions that may require immediate response during the communication lag period between feedback transmission and control command reception.

During operation, both the growing rod 20 and the external controller 22 can monitor similar operational parameters, with the external controller 22 receiving feedback data through the communication protocol while the growing rod 20 simultaneously monitors the same parameters locally through its main controller 104. When rapid changes occur in motor current, voltage levels, or other operational conditions, the growing rod 20 can implement immediate corrective actions using its redundant control capabilities without waiting for updated instructions from the external controller 22.

In one embodiment, both the external controller 22 and the growing rod 20 can implement a voltage regulation control function. The external controller 22 can monitor the voltage level of the motor 34 via the feedback signal, while the growing rod 20 can monitor the same voltage level via the electronics package 102. If, when the external controller 22 is adjusting the transmitted power signal amplitude based on the feedback signal, the main controller 104 identifies a voltage anomaly (e.g., excessively high or low voltage) that is not being addressed by the external controller 22, the main controller 104 can immediately correct the voltage anomaly without requiring any instruction from the external controller 22. This configuration can allow for an instantaneous response to the voltage anomaly. Once the voltage anomaly has been corrected, the external controller 22 can recognize the adjustment as a function of the feedback data and can adjust the power signal accordingly.

In another embodiment, both the external controller 22 and the growing rod 20 can implement a current regulation control function. The external controller 22 can monitor the current level of the motor 34 via the feedback signal, while the growing rod 20 can monitor the same current level via the electronics package 102. If, when the external controller 22 is adjusting the transmitted power signal amplitude based on the feedback signal, the main controller 104 identifies a current anomaly (e.g., excessively high or low voltage) that is not being addressed by the external controller 22, the main controller 104 can immediately correct the current anomaly without requiring any instruction from the external controller 22. This configuration can allow for an instantaneous response to the current anomaly. Once the current anomaly has been corrected, the external controller 22 can recognize the adjustment as a function of the feedback data and can adjust the power signal accordingly.

Additional control functions that can be controlled with the distributed control architecture can include temperature regulation where both devices monitor thermal conditions and implement cooling protocols, force limiting where both systems can independently restrict motor torque to prevent excessive distraction forces, and position control where both the external controller 22 and growing rod 20 can verify and correct extension rod positioning to maintain accurate lengthening distances. The system may also implement redundant safety shutdown procedures where either device can independently terminate operations upon detecting fault conditions such as mechanical binding, communication failures, or component malfunctions.

The redundant control architecture enables seamless operation where the growing rod 20 temporarily assumes control responsibilities normally handled by the external controller 22 during communication lag periods. Once the external controller 22 receives updated feedback data and transmits new control commands, the growing rod 20 can transition back to following external control instructions while maintaining its redundant capabilities in standby mode for future autonomous operation needs.

In some instances, the redundant control systems may implement synchronized operation where both devices execute the same control algorithms simultaneously, with the growing rod 20 serving as a backup control system that activates when communication latency exceeds predetermined thresholds. This synchronized redundancy ensures smooth transitions between external and local control modes without operational disruptions.

This distributed architecture with redundant control capabilities provides enhanced system reliability by ensuring that critical control functions continue uninterrupted even during temporary communication delays or coupling variations. As such, the growing rod 20 can maintain optimal operational parameters through its redundant control systems while the external controller 22 processes feedback data and formulates updated control strategies. The coordination between redundant control systems enables the system 23 to achieve consistent performance despite the inherent delays in wireless communication protocols. Furthermore, the distributed architecture enables the system 23 to achieve performance levels comparable to wired control systems while maintaining the clinical advantages of wireless operation, particularly in applications where communication delays could otherwise compromise treatment precision or safety during critical operational phases.

A method for wirelessly adjusting the growing rod 20 with the external controller 22 will now be described. First, a user (e.g., a medical professional) can input the distance that the growing rod 20 is to be lengthened, or other control parameters for the growing rod 20, into the external controller 22 using the display 32. The external controller 22 can then be positioned over the patient for alignment with the growing rod 20. Since the growing rod 20 is under the patient's skin, visually aligning the external controller 22 with the growing rod 20 can be difficult. Therefore, during alignment, the growing rod 20 and the external controller 22 can communicate with each other to detect the proximity of the growing rod 20 as a function of the impedance of the communication signal therebetween. The display 32 can provide visual feedback to the user that helps the user align the external controller 22 with the growing rod 20. Once the external controller 22 is aligned with the growing rod 20 (i.e., enough to facilitate communication and power delivery), the external controller 22 can tailor the frequency of the power signal to match the resonant frequency of the growing rod 20. The frequency of the power signal can be matched to the resonant frequency by conducting a frequency sweep procedure. Once the frequency of the power signal has been matched to the resonant frequency of the growing rod 20, the growing rod 20 can then be adjusted using the power and instructions from the power signal. During adjustment of the growing rod 20, the frequency sweep procedure can be periodically performed to accommodate for any changes to the position of the external controller 22 relative to the growing rod 20 (e.g., due to slight changes in the position of the external controller 22 as a result of being manually held in place by the user).

The power delivered by the power signal can also be adjusted periodically during adjustment of the growing rod 20 to accommodate for any physical and/or electrical variations that might occur. In some embodiments, the adjustment of the power signal can be achieved by: 1.) adjusting the voltage and/or motor control of the growing rod 20; 2.) controlling the voltage of the power signal; and/or 3.) dynamically adjusting the high/low duty for ASK (e.g., dynamic buck modulation). During each of these procedures, relevant information about the growing rod 20 can be provided to the external controller 22 via the feedback signal.

Referring now to FIG. 15, the external controller 22 is shown to be in communication with a remote computing environment 170 to allow for the transmission of data therebetween. The remote computing environment 170 can include a personal computer 172 (e.g., a desktop or a laptop), a smart device 174 (e.g., a smartphone or a tablet), a database 176, and a cloud 178. The personal computer 172, the smart device 174, the database 176, and the cloud 178 can operate independently or in conjunction with each other to analyze the data received from the external controller 22. In some embodiments, the personal computer 172 and/or the smart device 174 can display the data received from the external controller 22 in real time and, in some cases, can effectively serve as a clone of the display 32. In some instances, the external controller 22 can be controlled from the cloned device (e.g., the personal computer 172 and/or the smart device 174). A cloud-based web application or mobile client loaded on any of the devices of the remote computing environment can additionally or alternatively be provided that may be used to interact with the growing rod 20.

The external controller 22 and the remote computing environment 170 can be in communication with each other via a wireless interface (e.g., via WiFi, Bluetooth, or an RFID data transfer protocol) or wired interface. Similarly, the devices of the remote computing environment 170 can be in communication with each other via a wireless or wired interface. In some instances, the data from the external controller 22 can be stored on a portable storage device (e.g., an SD card or a thumb drive) that can be physically installed on one or more of the devices of the remote computing environment 170 to facilitate the transmission of the external controller data thereto.

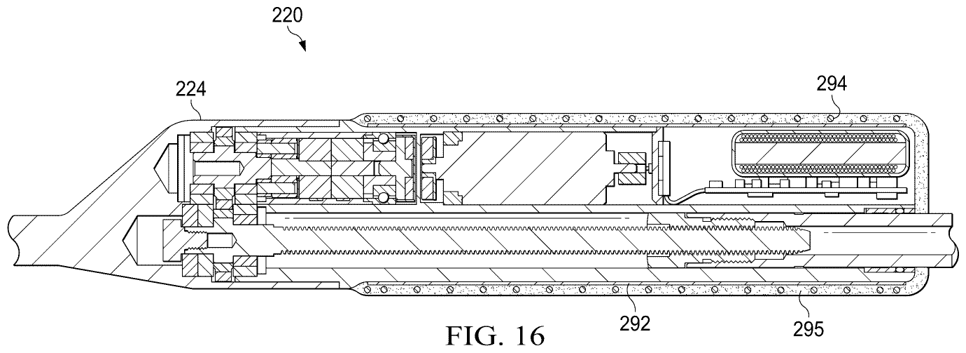

An alternative embodiment of a growing rod 220 is illustrated in FIG. 16 and can be similar to, or the same in many respects as, the growing rod 20 illustrated in FIGS. 1-11. For example, the growing rod 220 can include a main body 224, a ferrous core 292, and a receive antenna 294. However, the ferrous core 292 and the receive antenna 294 can be routed over, and can surround, the main body 224. The ferrous core 292 and the receive antenna 294 can be embedded in an epoxy layer 295.

The present disclosure provides an implantable medical device comprising: a main body; a motor having an output shaft; a gear box comprising: an input shaft operably coupled to the output shaft of the motor; and an output shaft operably coupled to the input shaft, the input shaft and the output shaft of the gear box being rotatable about a first axis; a drive gear coupled to the output shaft of the gear box and rotatable about the first axis; a lead screw rotatable about a second axis that is parallel to and axially offset from the first axis; a driven gear coupled to the lead screw and intermeshed with the drive gear such that rotation of the drive gear causes rotation of the lead screw; and an extension rod translatably coupled to the lead screw such that rotation of the lead screw causes the extension rod to slide along the second axis.

Optionally, the extension rod is threadedly engaged with the lead screw.

Optionally, the extension rod includes a nut formed of PEEK that is threadedly engaged with the lead screw.

Optionally, at least one of the driven gear and the drive gear are formed of PEEK.

Optionally, the motor is disposed in a first compartment and the gear box is disposed in a second compartment, and wherein the first compartment and the second compartment are separated by a wall; and the output shaft of the motor includes a plurality of magnets and the input shaft of the gear box includes a plurality of magnets, and wherein the magnets of the output shaft and the magnets of the input shaft are magnetically coupled together through the wall.

Optionally, the first compartment is hermetically sealed.

Optionally, the implantable medical device further comprises a sealing arrangement disposed between the main body and the extension rod.

Optionally, the sealing arrangement includes one or more of a bushing and an O-ring.

Optionally, the gear box includes a plurality of reduction stages that define a gear ratio between the input shaft and the output shaft of the gear box.

Optionally, the gear ratio is approximately 1466:1.

The present disclosure provides an implantable medical device comprising: a main body; a motor disposed within the main body; an extension rod operably coupled to the motor and configured to slide relative to the main body in response to operation of the motor; and a receive antenna configured to receive a power signal from an external controller, the power signal providing electrical power for operating the motor, wherein the implantable medical device is devoid of an onboard power storage device and is configured to operate solely from power received via the power signal from the external controller.

Optionally, the implantable medical device further comprises a dual antenna assembly including the receive antenna and a transmit antenna configured to transmit a feedback signal to the external controller.

Optionally, the dual antenna assembly includes a ferrous core, and wherein the receive antenna and the transmit antenna share the ferrous core.

Optionally, the receive antenna includes windings wound circumferentially around the ferrous core and the transmit antenna includes windings wound longitudinally around the ferrous core, such that the windings of the receive antenna and the windings of the transmit antenna are substantially orthogonal to each other.

Optionally, at least a portion of the receive antenna is sandwiched between the transmit antenna and the ferrous core.

Optionally, the receive antenna is configured to receive the power signal simultaneously while the transmit antenna transmits the feedback signal.

Optionally, the implantable medical device further comprises a main controller configured to extract communication data from the power signal and to separate electrical power from the communication data for operating the motor.

Optionally, the main controller includes a rectifier configured to demodulate the power signal to extract both the electrical power and the communication data.

Optionally, the implantable medical device further comprises: a gear box operably coupled to the motor and including an input shaft and an output shaft, the input shaft and the output shaft of the gear box being rotatable about a first axis; a lead screw rotatable about a second axis that is parallel to and axially offset from the first axis; and a drive mechanism coupling the gear box to the lead screw such that the extension rod is operably coupled to the lead screw.

Optionally, the motor includes an output shaft having a plurality of magnets and rotatable about the first axis, wherein the input shaft of the gear box includes a plurality of magnets, and wherein the magnets of the motor output shaft and the magnets of the input shaft of the gear box are magnetically coupled together.

Optionally, the drive mechanism includes a drive gear coupled to the output shaft of the gear box and a driven gear coupled to the lead screw, the drive gear and driven gear being intermeshed to transfer rotational motion from the first axis to the second axis.

Optionally, the main body defines a first compartment housing the motor and a second compartment housing the receive antenna, and wherein the motor compartment is hermetically sealed from the electronics compartment.

Optionally, the implantable medical device further comprises an encoder configured to monitor a rotational position of the motor and to provide position feedback data for transmission to the external controller.

Optionally, the extension rod includes a tapered portion and is movable to a sterilization position in which the tapered portion creates a flow path for sterilizing gas to enter internal compartments of the implantable medical device.

Optionally, the power signal is modulated to carry communication data, and wherein the implantable medical device is configured to extract both power and the communication data from the same modulated power signal.

The present disclosure provides a method for frequency optimization between an external controller and an implantable medical device, the method comprising: establishing a baseline frequency range for a power signal to be transmitted from the external controller to the implantable medical device; conducting a frequency sweep by transmitting test signals at discrete frequency steps within the baseline frequency range; monitoring feedback data received from the implantable medical device during the frequency sweep, the feedback data including power reception efficiency metrics; analyzing the feedback data to identify a frequency that provides a desired power transfer efficiency; and configuring the external controller to transmit the power signal at the identified frequency.

Optionally, establishing the baseline frequency range comprises determining an expected resonant frequency of a receive antenna within the implantable medical device based on manufacturing specifications or previous calibration procedures.

Optionally, the baseline frequency range is between about 100 kHz and 500 kHz.

Optionally, the baseline frequency range is between about 290 kHz and 390 kHz.

Optionally, the feedback data further includes signal quality indicators, coupling strength measurements, received signal strength, power conversion efficiency, and signal-to-noise ratio measurements.

Optionally, conducting the frequency sweep comprises implementing adaptive step sizing by beginning with relatively large frequency steps to identify regions of improved coupling efficiency and reducing the step size in promising frequency ranges to achieve finer resolution.

Optionally, analyzing the feedback data comprises applying weighting factors to different metrics based on their relative importance for specific application or operating conditions.

Optionally, the method further comprises implementing stability criteria to prevent undesirable frequency changes by requiring that a new frequency demonstrate improved performance characteristics over a current operating frequency before initiating a frequency change.

The present disclosure provides a system for controlling an implantable medical device using a distributed control architecture, comprising: an external controller configured to transmit control commands to an implantable medical device; and an implantable medical device having a main controller configured to receive the control commands and to monitor operational parameters of the implantable medical device; wherein both the external controller and the implantable medical device implement redundant control functions; wherein the external controller is configured to monitor operational parameters via feedback signals received from the implantable medical device; wherein the implantable medical device is configured to monitor operational parameters locally; and wherein the implantable medical device is configured to detect an anomaly in the operational parameters that is not being addressed by the external controller and to implement corrective action using the redundant control functions without waiting for instructions from the external controller.

Optionally, the operational parameters monitored include one or more of motor current levels, voltage levels across components, internal temperature readings, motor rotational position, extension rod position, power reception efficiency, signal quality indicators, and force measurements.

Optionally, the redundant control functions include one or more of voltage regulation control, current regulation control, temperature regulation control, force limiting control, position control, and safety shutdown procedures.

Optionally, the anomaly comprises one or more of excessively high or low voltage conditions, motor overcurrent conditions, internal temperature readings exceeding safety thresholds, motor position encoder failures, power reception efficiency below acceptable levels, motor stall conditions, and force measurements exceeding predetermined safety limits.

Optionally, the corrective action comprises one or more of adjusting voltage levels, modifying current delivery, implementing cooling protocols, restricting motor torque, correcting extension rod positioning, and initiating emergency shutdown procedures.

Optionally, the external controller is configured to wirelessly power the implantable medical device via a power signal, and wherein the power signal is modulated to carry the control commands.

Optionally, the implantable medical device is configured to transmit the feedback signals wirelessly to the external controller through a separate communication channel than power signal reception.

Optionally, the system is configured to automatically transition between external control mode and local control mode based on communication latency thresholds, and wherein the implantable medical device is configured to assume control responsibilities when communication delays exceed predetermined limits.

The present disclosure provides a method for controlling an implantable medical device using a distributed control architecture, comprising: providing an external controller configured to transmit control commands to an implantable medical device; providing an implantable medical device having a main controller configured to receive the control commands and to monitor operational parameters of the implantable medical device; implementing redundant control functions on both the external controller and the implantable medical device; monitoring operational parameters at the external controller via feedback signals; monitoring operational parameters at the implantable medical device; and when the implantable medical device detects an anomaly in the operational parameters that is not being addressed by the external controller, implementing corrective action at the implantable medical device using the redundant control functions without waiting for instructions from the external controller.

Optionally, the operational parameters monitored include one or more of motor current levels, voltage levels across components, internal temperature readings, motor rotational position, extension rod position, power reception efficiency, signal quality indicators, and force measurements.

Optionally, the redundant control functions include one or more of voltage regulation control, current regulation control, temperature regulation control, force limiting control, position control, and safety shutdown procedures.

Optionally, the anomaly comprises one or more of excessively high or low voltage conditions, motor overcurrent conditions, internal temperature readings exceeding safety thresholds, motor position encoder failures, power reception efficiency below acceptable levels, motor stall conditions, and force measurements exceeding predetermined safety limits.

Optionally, the corrective action comprises one or more of adjusting voltage levels, modifying current delivery, implementing cooling protocols, restricting motor torque, correcting extension rod positioning, and initiating emergency shutdown procedures.

Optionally, the implantable medical device is wirelessly powered by a power signal transmitted from the external controller, and wherein the power signal is modulated to carry the control commands.

Optionally, the implantable medical device transmits the feedback signals wirelessly to the external controller through a separate communication channel than the power signal reception.

Optionally, the method further comprises automatically transitioning between external control mode and local control mode based on communication latency thresholds, wherein the implantable medical device assumes control responsibilities when communication delays exceed predetermined limits.

The present disclosure provides a system for treating a medical condition, the system comprising: an implantable medical device configured to be implanted in a patient, the implantable medical device including: a main body defining a first compartment and a second compartment; a motor disposed in the first compartment; an extension rod slidably coupled with the main body; and a dual antenna assembly disposed in the second compartment and comprising a receive antenna for receiving a power signal and a transmit antenna for transmitting a feedback signal; and an external controller configured to wirelessly communicate with the implantable medical device, the external controller including: a housing; a dual antenna assembly comprising: a transmit antenna for transmitting the power signal with communication data embedded therein to the implantable medical device, and a receive antenna for receiving the feedback signal from the implantable medical device, wherein the external controller is configured to wirelessly power the implantable medical device via the power signal and to receive operational feedback from the implantable medical device via the feedback signal.

Optionally, the dual antenna assembly of the implantable medical device includes a ferrous core, and wherein the receive antenna and the transmit antenna of the implantable medical device share the ferrous core.

Optionally, the receive antenna of the implantable medical device includes windings wound circumferentially around the ferrous core and the transmit antenna of the implantable medical device includes windings wound longitudinally around the ferrous core, such that the windings of the receive antenna and the windings of the transmit antenna are substantially orthogonal to each other.

Optionally, at least a portion of the receive antenna of the implantable medical device is sandwiched between the transmit antenna and the ferrous core.

Optionally, the dual antenna assembly of the external controller includes a ferrous core and a bobbin, the ferrous core including a central portion and a pair of leg portions extending from the central portion.

Optionally, the transmit antenna of the external controller includes windings wound circumferentially around the central portion of the ferrous core and the receive antenna of the external controller includes windings wound circumferentially around an outer perimeter of the bobbin.

Optionally, the leg portions of the ferrous core extend into receptacles of the bobbin such that the ferrous core and the bobbin form a unitary core, and wherein the windings of the transmit antenna and the windings of the receive antenna of the external controller are substantially orthogonal to each other.

Optionally, the implantable medical device further comprises a main controller configured to extract communication data from the power signal and to separate electrical power from the communication data for operating the motor.

Optionally, the power signal is modulated to carry the communication data, and wherein the implantable medical device is configured to extract both power and the communication data from the same modulated power signal.

Optionally, the external controller is configured to conduct a frequency sweep by transmitting test signals at discrete frequency steps within a baseline frequency range and to monitor feedback data received from the implantable medical device during the frequency sweep.

Optionally, the external controller is configured to analyze the feedback data to identify a frequency that provides a desired power transfer efficiency and to set the power signal transmission at the identified frequency.

The present disclosure provides an implantable medical device comprising: a main body defining a first compartment; an extension rod slidably coupled with the main body and at least partially disposed in the first compartment, the extension rod being movable between a retracted position, an extended position, and a sterilization position; a sealing arrangement disposed between the main body and the extension rod; wherein: the extension rod includes a proximal end, a distal end, and a tapered portion disposed between the proximal end and the distal end; and when the extension rod is in the sterilization position, the tapered portion is withdrawn from the sealing arrangement to create a flow path between the extension rod and the sealing arrangement that allows sterilizing gas to flow into the first compartment.

Optionally, the sealing arrangement includes one or more of a bushing and an O-ring.

Optionally, the implantable medical device further comprises a gear box disposed in a second compartment and operably coupled with the extension rod, wherein the second compartment is in fluid communication with the first compartment to receive sterilizing gas introduced into the first compartment through the flow path.

Optionally, the implantable medical device further comprises: a motor having an output shaft rotatable about a first axis; a gear box having an input shaft and an output shaft rotatable about the first axis; and a lead screw rotatable about a second axis that is parallel to and axially offset from the first axis, the lead screw being operably coupled to the extension rod.

Optionally, the implantable medical device further comprises a drive gear coupled to the output shaft of the gear box and rotatable about the first axis, and a driven gear coupled to the lead screw and intermeshed with the drive gear such that rotation of the drive gear causes rotation of the lead screw about the second axis.

Optionally, the extension rod is threadedly engaged with the lead screw such that rotation of the lead screw causes the extension rod to slide along the second axis.

Optionally, the implantable medical device further comprises a receive antenna configured to receive a power signal from an external controller, wherein the implantable medical device is configured to be wirelessly controlled by the external controller to move the extension rod to the sterilization position.

Optionally, the power signal is modulated to carry communication data that includes commands for positioning the extension rod in the sterilization position, and wherein the implantable medical device is configured to extract both power and the communication data from the modulated power signal.

The present disclosure provides a method for sterilizing an implantable medical device, comprising: providing an implantable medical device having an extension rod that is at least partially disposed in a compartment of the implantable medical device and that is movable between a starting position and a sterilization position, the extension rod including a tapered portion; positioning the extension rod in the sterilization position such that the tapered portion creates a flow path for sterilizing gas to enter the compartment of the implantable medical device; placing the implantable medical device in a sealable pouch; introducing sterilizing gas into the sealable pouch such that the sterilizing gas flows through the flow path into the compartment; maintaining the sterilizing gas in contact with surfaces within the internal compartments for a predetermined time; and moving the extension rod from the sterilization position to the starting position after sterilization is complete.

Optionally, the tapered portion is disposed between a proximal end and a distal end of the extension rod, and wherein the tapered portion tapers inwardly towards the distal end.

Optionally, the sterilizing gas comprises one or more of ethylene oxide, chlorine dioxide, and hydrogen peroxide.

Optionally, positioning the extension rod in the sterilization position comprises transmitting control commands from the external controller to the implantable medical device to move the extension rod to the sterilization position.

Optionally, the external controller wirelessly transmits the control commands to the implantable medical device and wirelessly powers the implantable medical device during positioning of the extension rod in the sterilization position.

Optionally, the external controller transmits a power signal that is modulated to carry communication data including the control commands for positioning the extension rod in the sterilization position.

Optionally, the method further comprises restricting access to move the extension rod to the sterilization position to authorized users through authentication mechanisms that verify user credentials before enabling sterilization positioning commands.

Optionally, the method further comprises evacuating the sterilizing gas from the sealable pouch through a controlled venting process that includes multiple evacuation and purge cycles to remove residual sterilizing agent.

Optionally, the implantable medical device includes a sealing arrangement disposed between the main body and the extension rod; the flow path is created by withdrawing the tapered portion from the sealing arrangement when the extension rod is in the sterilization position; and the flow path is blocked by interfacing the tapered portion with the sealing arrangement when the extension rod is in the sterilizing position.

The present disclosure provides an implantable medical device, comprising: a main body defining a first compartment and a second compartment and comprising a wall that separates the first compartment from the second compartment; a motor disposed in the first compartment and having an output shaft and a rotary magnet operably coupled to the output shaft; and an encoder disposed in the second compartment and including a base plate and a plurality of magnets arranged radially about the base plate, the encoder being positioned adjacent to the wall such that the plurality of magnets detect a position of the rotary magnet through the wall to facilitate monitoring of a position of the output shaft.

Optionally, the encoder may be implemented as one of a magnetic-based encoder, a reflected light encoder utilizing a ceramic PCB with an encoder disc having reflective engravings, a transmitted light encoder with cutouts in an encoder disc positioned between emitters and photo detectors, or an optical motor encoder with emitter/receiver packages for position, speed, and direction detection.

Optionally, the first compartment is hermetically sealed to prevent infiltration of environmental contaminants.

Optionally, the implantable medical device further comprises a transmit antenna configured to transmit feedback data including position information from the encoder to an external controller, wherein the implantable medical device is configured to be wirelessly controlled based on the position feedback from the encoder.

Optionally, the implantable medical device further comprises a receive antenna configured to receive control commands from the external controller, wherein the control commands include instructions for motor positioning based on the encoder feedback data.

Optionally, the implantable medical device implements closed-loop position control by transmitting real-time encoder position data to the external controller and receiving position correction commands in response to the transmitted position data.

Optionally, the implantable medical device further comprises a receive antenna configured to receive a power signal from an external controller, wherein the implantable medical device is devoid of an onboard power storage device and operates solely from power received via the power signal.

Optionally, the power signal is modulated to carry communication data; and the implantable medical device is configured to extract both electrical power and the communication data from the same modulated power signal.

Optionally, when one of the plurality of magnets experiences degradation or failure, at least one of the remaining magnets continue to provide position or speed feedback; and a controller associated with the encoder is configured to automatically adjust calculation algorithms to account for the degradation or failure of the at least one remaining magnets while maintaining adequate resolution for motor control purposes.

The present disclosure provides a method for collecting and utilizing data from an implantable medical device, the method comprising: collecting feedback data from an implantable medical device during treatment sessions, the feedback data including operational parameters of the implantable medical device; logging the feedback data; analyzing the logged feedback data; and generating treatment recommendations for future medical procedures based on the analyzed feedback data.

Optionally, analyzing the logged feedback data comprises identifying patterns in mechanical resistance at specific extension distances across multiple treatment sessions; and generating treatment recommendations comprises suggesting modified extension protocols based on the identified patterns.

Optionally, the treatment recommendations include gradual force ramping, extended dwell times at resistance points, or adjusted extension speeds to accommodate the identified patterns.

Optionally, the operational parameters include motor current measurements, force application profiles, extension speeds, and temperature readings, and wherein analyzing the logged feedback data comprises correlating motor current measurements with actual force output to establish baseline operational parameters for future treatment sessions.

Optionally, generating treatment recommendations comprises implementing predictive modeling using machine learning algorithms to forecast optimal treatment parameters for upcoming sessions based on historical feedback patterns, the treatment recommendations including suggested extension distances, force application profiles, and safety thresholds customized to individual patient biomechanical characteristics.

Optionally, the method further comprises transmitting the treatment recommendations to an external controller or a remote computing device for presentation to a medical practitioner.

Optionally, analyzing the logged feedback data comprises detecting trends in patient biomechanical response over time; and generating treatment recommendations comprises automatically adjusting treatment session timing, extension protocols, and force application strategies based on the detected trends.

The present disclosure provides a method for data logging and treatment optimization for an implantable medical device, comprising: collecting feedback data from an implantable medical device during treatment sessions, the feedback data including operational parameters of the implantable medical device; logging the feedback data; analyzing the logged feedback data; and implementing self-calibration of the implantable medical device by automatically adjusting operational parameters based on the logged feedback data to compensate for changes in component behavior over time.

Optionally, implementing self-calibration comprises analyzing historical motor current measurements to identify gradual changes in motor efficiency and automatically updating motor control parameters to maintain consistent torque output despite component aging or mechanical wear.

Optionally, implementing self-calibration comprises monitoring position encoder readings over multiple treatment sessions to detect systematic offset errors and automatically adjusting position scaling factors to maintain accurate extension distance measurements.

Optionally, implementing self-calibration comprises correlating temperature data patterns with operational performance metrics to automatically update thermal compensation parameters that account for temperature variations affecting device performance.

Optionally, implementing self-calibration comprises comparing actual operational parameters against baseline values stored during initial device calibration and automatically generating calibration adjustment coefficients when systematic deviations exceed predetermined thresholds.

Optionally, implementing self-calibration comprises transmitting updated calibration parameters from an external controller to the implantable medical device through wireless communication, and wherein the implantable medical device stores the updated parameters in internal memory for application during subsequent operations.

Optionally, implementing self-calibration comprises executing calibration adjustments automatically at predetermined intervals based on accumulated treatment session data or when statistical significance thresholds are reached.

Optionally, implementing self-calibration comprises incorporating machine learning algorithms that continuously refine calibration parameters based on ongoing data collection to adapt to changing conditions throughout an extended implantation period.

Optionally, implementing self-calibration comprises generating calibration logs that document parameter adjustments, detected performance deviations, and recommended calibration intervals for review by medical practitioners.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein but can be employed in any number of applications and equivalent devices by those of ordinary skill in the art. Rather, it is hereby intended that the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

1. An implantable medical device comprising:
a main body;
a motor having an output shaft;
a gear box comprising:
an input shaft operably coupled to the output shaft of the motor; and
an output shaft operably coupled to the input shaft, the input shaft and the output shaft of the gear box being rotatable about a first axis;
a drive gear coupled to the output shaft of the gear box and rotatable about the first axis;
a lead screw rotatable about a second axis that is parallel to and axially offset from the first axis;
a driven gear coupled to the lead screw and intermeshed with the drive gear such that rotation of the drive gear causes rotation of the lead screw; and
an extension rod translatably coupled to the lead screw such that rotation of the lead screw causes the extension rod to slide along the second axis.

2-4. (canceled)

5. The implantable medical device of claim 1, wherein:
the motor is disposed in a first compartment and the gear box is disposed in a second compartment, and wherein the first compartment and the second compartment are separated by a wall; and
the output shaft of the motor includes a plurality of magnets and the input shaft of the gear box includes a plurality of magnets, and wherein the magnets of the output shaft and the magnets of the input shaft are magnetically coupled together through the wall.

6-10. (canceled)

11. An implantable medical device comprising:
a main body;
a motor disposed within the main body;
an extension rod operably coupled to the motor and configured to slide relative to the main body in response to operation of the motor; and
a receive antenna configured to receive a power signal from an external controller, the power signal providing electrical power for operating the motor, wherein the implantable medical device is devoid of an onboard power storage device and is configured to operate solely from power received via the power signal from the external controller.

12. The implantable medical device of claim 11, further comprising a dual antenna assembly including the receive antenna and a transmit antenna configured to transmit a feedback signal to the external controller.

13. The implantable medical device of claim 12, wherein the dual antenna assembly includes a ferrous core, and wherein the receive antenna and the transmit antenna share the ferrous core.

14. The implantable medical device of claim 13, wherein the receive antenna includes windings wound circumferentially around the ferrous core and the transmit antenna includes windings wound longitudinally around the ferrous core, such that the windings of the receive antenna and the windings of the transmit antenna are substantially orthogonal to each other.

15-18. (canceled)

19. The implantable medical device of claim 11, further comprising:
a gear box operably coupled to the motor and including an input shaft and an output shaft, the input shaft and the output shaft of the gear box being rotatable about a first axis;
a lead screw rotatable about a second axis that is parallel to and axially offset from the first axis; and
a drive mechanism coupling the gear box to the lead screw such that the extension rod is operably coupled to the lead screw.

20. The implantable medical device of claim 19, wherein the motor includes an output shaft having a plurality of magnets and rotatable about the first axis, wherein the input shaft of the gear box includes a plurality of magnets, and wherein the magnets of the motor output shaft and the magnets of the input shaft of the gear box are magnetically coupled together.

21. The implantable medical device of claim 19, wherein the drive mechanism includes a drive gear coupled to the output shaft of the gear box and a driven gear coupled to the lead screw, the drive gear and driven gear being intermeshed to transfer rotational motion from the first axis to the second axis.

22-60. (canceled)

61. An implantable medical device comprising:
a main body defining a first compartment;
an extension rod slidably coupled with the main body and at least partially disposed in the first compartment, the extension rod being movable between a retracted position, an extended position, and a sterilization position;
a sealing arrangement disposed between the main body and the extension rod; wherein:
the extension rod includes a proximal end, a distal end, and a tapered portion disposed between the proximal end and the distal end; and
when the extension rod is in the sterilization position, the tapered portion is withdrawn from the sealing arrangement to create a flow path between the extension rod and the sealing arrangement that allows sterilizing gas to flow into the first compartment.

62. (canceled)

63. The implantable medical device of claim 61, further comprising a gear box disposed in a second compartment and operably coupled with the extension rod, wherein the second compartment is in fluid communication with the first compartment to receive sterilizing gas introduced into the first compartment through the flow path.

64. The implantable medical device of claim 61, further comprising:
a motor having an output shaft rotatable about a first axis;
a gear box having an input shaft and an output shaft rotatable about the first axis; and
a lead screw rotatable about a second axis that is parallel to and axially offset from the first axis, the lead screw being operably coupled to the extension rod.

65. The implantable medical device of claim 64, further comprising a drive gear coupled to the output shaft of the gear box and rotatable about the first axis, and a driven gear coupled to the lead screw and intermeshed with the drive gear such that rotation of the drive gear causes rotation of the lead screw about the second axis.

66. The implantable medical device of claim 64, wherein the extension rod is threadedly engaged with the lead screw such that rotation of the lead screw causes the extension rod to slide along the second axis.

67. The implantable medical device of claim 61, further comprising a receive antenna configured to receive a power signal from an external controller, wherein the implantable medical device is configured to be wirelessly controlled by the external controller to move the extension rod to the sterilization position.

68. The implantable medical device of claim 67, wherein the power signal is modulated to carry communication data that includes commands for positioning the extension rod in the sterilization position, and wherein the implantable medical device is configured to extract both power and the communication data from the modulated power signal.

69. A method for sterilizing an implantable medical device, comprising:
providing an implantable medical device having an extension rod that is at least partially disposed in a compartment of the implantable medical device and that is movable between a starting position and a sterilization position, the extension rod including a tapered portion;
positioning the extension rod in the sterilization position such that the tapered portion creates a flow path for sterilizing gas to enter the compartment of the implantable medical device;
placing the implantable medical device in a sealable pouch;
introducing sterilizing gas into the sealable pouch such that the sterilizing gas flows through the flow path into the compartment;
maintaining the sterilizing gas in contact with surfaces within the internal compartments for a predetermined time; and
moving the extension rod from the sterilization position to the starting position after sterilization is complete.

70. The method of claim 69, wherein the tapered portion is disposed between a proximal end and a distal end of the extension rod, and wherein the tapered portion tapers inwardly towards the distal end.

71-73. (canceled)

74. The method of claim 69, wherein the external controller transmits a power signal that is modulated to carry communication data including the control commands for positioning the extension rod in the sterilization position.

75. The method of claim 69, further comprising restricting access to move the extension rod to the sterilization position to authorized users through authentication mechanisms that verify user credentials before enabling sterilization positioning commands.

76-102. (canceled)
